(19) United States
(12) Patent Application Publication
(10) Pub. No.: US 2015/0293065 A1
(43) Pub. Date: Oct. 15, 2015

(54) Coatings with nanomaterials

(71) Applicants: Kyle R. Kissell, Manvel, TX (US); William D. Stringfellow, Houston, TX (US); John B. Stuart, Kingwood, TX (US); Guy L. McClung, III, San Antonio, TX (US)

(72) Inventors: Kyle R. Kissell, Manvel, TX (US); William D. Stringfellow, Houston, TX (US); John B. Stuart, Kingwood, TX (US); Guy L. McClung, III, San Antonio, TX (US)

(21) Appl. No.: 13/999,995

(22) Filed: Apr. 11, 2014

(51) Int. Cl. G01N 33/00; G01M 99/00

(52) U.S. Cl. CPC G01N 33/00; G01M 99/00; G01N 2033/0096

(57) ABSTRACT

Coatings and heatable coatings containing nanomaterial, which in on aspect is electrically conductive nanomaterial; methods for making such a coating; items with such a coating; and methods for applying such a coating. In one aspect, such a coating is a deicing coating. This abstract is provided to comply with the rules requiring an abstract which will allow a searcher or other reader to quickly ascertain the subject matter of the technical disclosure and is submitted with the understanding that it will not be used to interpret or limit the scope or meaning of the claims, 37 C.F.R. 1.72(b).

### RELATED APPLICATION

This is a continuation-in-part of U.S. application Ser. No. 13/998,093 filed Sep. 30, 2013 which is a division of U.S. application Ser. No. 12/924,729 filed Oct. 4, 2010; and the present invention and application claim priority under the Patent Laws from said applications, which applications are incorporated fully herein for all purposes.

### BACKGROUND OF THE INVENTION

1. Field of the Invention

The present invention is directed to protective coatings, to heatable coatings, to deicing coatings containing electrically conductive nanomaterials, methods for making them, methods for heating such coatings, methods for deicing, items with such coatings and, in certain aspects, to such coatings with carbon nanotubes therein.

2. Description of Related Art

There is a wide variety of known approaches to heating things and surfaces with heatable coatings and to dealing with ice formation on items and on surfaces, e.g., on of wings, propellers, turbine blades, ship hulls, paved areas, bridges, runways, train tracks, pipelines, refinery equipment and apparatuses, piping, petroleum and chemical plant apparatuses and flow conduits, and power towers; as well as ice formation on equipment and machines, including, but not limited to, equipment and machines used in the exploration for, drilling of and production from oil and gas wells and the processing of recovered gas and oil.

Prior patents and applications disclose a variety of articles, substances, devices, apparatuses, and methods for dealing with icing problems, including, but not limited to, those in, and those referred to in or cited in, exemplary U.S. Pat. Nos. 6,832,742; 6,790,526; 6,773,877; 6,576,115; 6,427,946; 6,303,388; 6,027,075; 4,737,618; 4,732,351; 4,685,967; 3,825,371; and 3,204,084 (all said patents incorporated fully herein for all purposes).

There have long been needs, recognized by the present inventors, for a durable and effective coating, for heatable coating; and for deicing material and methods of their use.

### SUMMARY OF THE PRESENT INVENTION

The present invention, in certain aspects, discloses a heatable coating with conductive nanomaterial. In certain aspects, such a coating, when heated resistively by the application of an electric current (AC or DC), heats an item or surface to which it has been applied; and, in particular circumstances, melts ice on items and on surfaces to which the coating has been applied. Such coatings can be used, among other things, to remediate paraffin wax in oil pipelines; to remediate or prevent hydrate formation or hydrate build-up in oil pipelines; and for viscosity control of heavy crude oil in storage tanks. A coating according to the present invention, any disclosed herein, may be a protective coating, a heatable coating, or a deicing coating according to the present invention, and an electrical system may be used with an event sensor or sensors which sense an event related to the item with the coating. For example, the sensed event may be a temperature increase, the formation of ice, or a change in temperature. Upon such sensing occurring, the electrical system changes the temperature of the coating or heats the coating, e.g. to deice the item.

In one aspect, such a coating has nanomaterials therein which are electrically conductive, for example, and not limited to, electrically conductive nanotubes, nanographene, nanographene ribbons, transformed nanomaterials (e.g., as in U.S. patent application Ser. Nos. 12/657,244; 12/657,288; 12/657,289—all filed on Jan. 16, 2010 and co-owned with the present invention and all incorporated fully herein for all purposes) and carbon nanomaterials, e.g., but not limited to, carbon nanotubes. In certain aspects, such a coating is between 0.0001 and 1.0 inches thick.

In one aspect, such a coating includes a resin material (e.g., but not limited to: any suitable known resin system; a one-part resin; a two-part resin; a one-component resin; and a two-component resin—any, optionally, with a catalyst and/or hardener). Sufficient nanomaterials are in the resin material so that upon heating of the coating, e.g., but not limited to, by the application of an electric current (DC or AC) to the coating, the coating is heated. In one aspect, the nanomaterial is mixed with the backing material, e.g., a resin material using suitable known mixing techniques, e.g., but not limited to, with a Banbury mixer or a Haake mixer or by known sonication mixing methods. In one aspect, the nanomaterial is nanotubes.

In one particular embodiment, the resin material is a combination of a polyisocyanate resin and a polyester resin. In certain aspects, an item according to the present invention includes a base or backing, e.g., a backing made of any suitable material to which the coating can be applied; including, but not limited to, glass, natural fabric, synthetic fabric, metals, elastomer, wood, plastics, composites, polymers, thermoplastics, thermosets, and in one particular aspect is HDPE (high density polyethylene) and a coating according to the present invention which includes coating material and an amount of carbon nanomaterial (nanotubes, nanoribbons, etc.) dispersed through the coating material. Any base, backing, or surface of an item according to the present invention may be prepped or prepared in any suitable known manner as is appropriate prior to the application of a coating according to the present invention. It is within the scope of the present invention for any known suitable coating material to be used to which sufficient nanomaterial can be added so that a conductivity level is reached that enables heating of the coating material. In certain aspects, the resistivity of a coating according to the present invention ranges between 1 to 100 g*.OMEGA./cm (which is also Ω/cm). In certain aspects, the amount of nanomaterial is less than 5% by weight of a coating. In certain aspects, the amount of nanomaterial is, by weight, 1% or 4%, and, in other aspects, the amount of nanomaterial ranges between 5% and 32% by weight. In other aspects, this amount is as low as 0.1% and can range between 0.1% and 5% by weight or between 0.1% and 32%. The base or backing may be a surface of a thing, including, but not limited to, a surface of a substrate.

In certain aspects, a resin system, a base, a surface, and/or a backing are chosen from known engineered materials that provide moisture resistance, ultraviolet light resistance, acid resistance, and base resistance. These have low permeability, low surface energy, high durability and high flexibility.

It is within the scope of the present invention for a coating according to the present invention to be applied in any suitable known manner of coating application, including, but not limited to, spraying, dipping, spreading, pouring, bonding, trowelling, application with a brush or roller, electrostatic coating, and fusion bonding. In multi-component coatings, it is within the scope of the present invention to mix together the components and then to apply the mixture; or to apply one, two, or more components separately in sequence or simultaneously, e.g., but not limited to, spraying different components with separate sprayers or spraying different components with a dual, triple, or more component feed system so that the mixture is sprayed from one exit port or nozzle. It is within the scope of the present invention to use any coating herein without the application of an electrical current to it and to provide any of the items and things disclosed herein with a coating according to the present invention without any voltage application apparatus or device.

Accordingly, the present invention includes features and advantages which are believed to enable it to advance coating technology, strengthening coatings, protective coatings, and heatable coating technology, and deicing technology. Characteristics and advantages of the present invention described above and additional features and benefits will be readily apparent to those skilled in the art upon consideration of the following description of preferred embodiments and referring to the accompanying drawings.

Certain embodiments of this invention are not limited to any particular individual feature disclosed here, but include combinations of them distinguished from the prior art in their structures, functions, and/or results achieved. Features of the invention have been broadly described so that the detailed descriptions of embodiments preferred at the time of filing for this patent that follow may be better understood, and in order that the contributions of this invention to the arts may be better appreciated. There are, of course, additional aspects of the invention described below and which may be included in the subject matter of the claims to this invention. Those skilled in the art who have the benefit of this invention, its teachings, and suggestions will appreciate that the conceptions of this disclosure may be used as a creative basis for designing other structures, methods and systems for carrying out and practicing the present invention. The claims of this invention are to be read to include any legally equivalent devices or methods which do not depart from the spirit and scope of the present invention.

What follows are some of, but not all, the objects of this invention. In addition to the specific objects stated below for at least certain embodiments of the invention, other objects and purposes will be readily apparent to one of skill in this art who has the benefit of this invention's teachings and disclosures. It is, therefore, an object of at least certain embodiments of the present invention to provide the embodiments and aspects listed above and:

New, useful, unique, efficient, nonobvious coatings with nanomaterial, heatable coatings, and deicing coatings with electrically conductive nanomaterials therein and methods for making them and applying them.

New, useful, unique, efficient, nonobvious deicing coatings with electrically conductive nanomaterials therein and method for making them and applying them.

New, useful, unique, efficient, nonobvious heatable coatings that include a backing and carbon nanotubes within the backing; and

New, useful, unique, efficient, nonobvious deicing coatings that include a backing and carbon nanotubes within the backing; and

New, useful, unique, efficient, nonobvious items with any such coating and methods for making and applying such coatings.

The present invention recognizes and addresses the problems and needs in this area and provides a solution to those problems and a satisfactory meeting of those needs in its various possible embodiments and equivalents thereof. To one of skill in this art who has the benefits of this invention's realizations, teachings, disclosures, and suggestions, various purposes and advantages will be appreciated from the following description of certain preferred embodiments, given for the purpose of disclosure, when taken in conjunction with the accompanying drawings. The detail in these descriptions is not intended to thwart this patent's object to claim this invention no matter how others may later attempt to disguise it by variations in form, changes, or additions of further improvements.

The Abstract that is part hereof is to enable the U.S. Patent and Trademark Office and the public generally, and scientists, engineers, researchers, and practitioners in the art who are not familiar with patent terms or legal terms of phraseology to determine quickly, from a cursory inspection or review, the nature and general area of the disclosure of this invention. The Abstract is neither intended to define the invention, which is done by the claims, nor is it intended to be limiting of the scope of the invention or of the claims in any way.

It will be understood that the various embodiments of the present invention may include one, some, or all of the disclosed, described, and/or enumerated improvements and/or technical advantages and/or elements in claims to this invention.

Certain aspects, certain embodiments, and certain preferable features of the invention are set out herein. Any combination of aspects or features shown in any aspect or embodiment can be used except where such aspects or features are mutually exclusive.

### BRIEF DESCRIPTION OF THE SEVERAL VIEWS OF THE DRAWINGS

A more particular description of embodiments of the invention briefly summarized above may be had by references to the embodiments which are shown in the drawings which form a part of this specification. These drawings illustrate embodiments preferred at the time of filing for this patent and are not to be used to improperly limit the scope of the invention which may have other equally effective or legally equivalent embodiments.

FIG. 1A is a schematic perspective view—not to scale—of a coating according to the present invention on an item or surface.

FIG. 1B is a graphic view of parameters of a system according to the present invention.

FIG. 2 is a schematic view of a system according to the present invention.

FIG. 3 is a schematic view of a system according to the present invention.

FIG. 4A is a top view of a plane according to the present invention.

FIG. 4B is a cross-section view of part of a wing of the plane of FIG. 4A.

FIG. 5 is a perspective view of a helicopter according to the present invention with coating according to the present invention.

FIG. 6 is a perspective view of a boat motor and propeller according to the present invention with coating according to the present invention.

FIG. 7A is a side view of a turbine blade according to the present invention with coating according to the present invention.

FIG. 7B is a side cross-section view of a turbine blade according to the present invention with coating according to the present invention.

FIG. 8A is a side view of a tower according to the present invention with coating according to the present invention.

FIG. 8B is a side view of a tower according to the present invention with coating according to the present invention.

FIG. 9 is a front view of a propeller according to the present invention with coating according to the present invention.

FIG. 10 is a perspective view of a wind power generator system according to the present invention with coating according to the present invention.

FIG. 11A is a schematic side view of a pipeline according to the present invention with coating according to the present invention

FIG. 11B is a schematic side view of a pipeline according to the present invention with coating according to the present invention.

FIG. 12 is a side view of a bridge according to the present invention with coating according to the present invention.

FIG. 13 is a side view of a ship according to the present invention with coating according to the present invention.

FIG. 14 is a schematic perspective view of a train according to the present invention with coating according to the present invention.

FIG. 15 is a perspective view of a rail according to the present invention with coating according to the present invention.

FIG. 16 is a side view of an automobile according to the present invention with coating according to the present invention.

FIG. 17 is a perspective view of a tractor trailer rig according to the present invention with coating according to the present invention.

FIG. 18 is a perspective view of a pick-up truck according to the present invention with coating according to the present invention.

FIG. 19 is a side view of a recreation vehicle according to the present invention with coating according to the present invention.

FIG. 20 is a side view of a travel trailer according to the present invention with coating according to the present invention.

FIG. 21 is a perspective view of a utility trailer according to the present invention with coating according to the present invention.

FIG. 22 is a schematic view of a drilling system according to the present invention with parts with coating according to the present invention.

FIG. 23 is a schematic view of a drilling system according to the present invention with parts with coating according to the present invention.

FIG. 24 is a schematic view of an offshore rig according to the present invention with parts with coating according to the present invention.

FIG. 25 is a side view of a blowout preventer system according to the present invention with parts with coating according to the present invention.

FIG. 26 is a cross-section view of a centrifuge according to the present invention with parts with coating according to the present invention.

FIG. 27 is a perspective view of a shale shaker according to the present invention with parts with coating according to the present invention.

FIG. 28 is a top view of a shale shaker screen according to the present invention with parts with coating according to the present invention.

FIG. 29 is a top view of a screen support according to the present invention with parts with coating according to the present invention.

FIG. 30 is a top view of a shale shaker screen according to the present invention with parts with coating according to the present invention.

FIG. 31 is a cross-section view of a shale shaker screen according to the present invention with parts with coating according to the present invention.

FIG. 32 is a perspective view of a heat exchanger according to the present invention with parts with coating according to the present invention.

FIG. 33 is a side view of a heat exchanger tube according to the present invention with parts with coating according to the present invention.

FIG. 34 is a perspective view of a jet engine according to the present invention with parts with coating according to the present invention.

FIG. 35 is a cross-section view of a tubular according to the present invention with parts with coating according to the present invention.

FIG. 36 is a cross-section view of a pipeline or conduit according to the present invention with parts with coating according to the present invention.

FIG. 37A is a partial cross-section view of a storage tank system as shown in FIG. 37B according to the present invention with parts with coating according to the present invention.

FIG. 37B is a top view partially cutaway of a storage tank system according to the present invention with parts with coating according to the present invention.

FIG. 38 is a side view partially in cross-section a pipeline pig according to the present invention with parts with coating according to the present invention.

FIG. 39 is a side cross-section view of fabric according to the present invention with parts with coating according to the present invention.

FIG. 40 is a side view of a blowout preventer according to the present invention with parts with coating according to the present invention.

FIG. 41A is a perspective view of a tubular according to the present invention.

FIG. 41B is a cross-section view of the tubular of FIG. 41A.

FIG. 42 is a cross-section view of a tubular according to the present invention.

FIG. 43 is a perspective view of a tubular according to the present invention.

FIG. 44 is a perspective view of a tubular according to the present invention.

FIG. 45 is a cross-section view of a tubular according to the present invention.

FIG. 46 is a perspective view of an item according to the present invention.

FIG. 47 is a perspective view of an item according to the present invention.

FIG. 48 is a perspective view of an item according to the present invention.

FIG. 49 is a schematic view of a system according to the present invention with a thing with a coating according to the present invention.

FIG. 50A is a schematic view of a thing coated with a coating according to the present invention.

FIG. 50B is a schematic view of one embodiment of a thing as in FIG. 50B with nanomaterial therein.

FIG. 51A is a schematic view of a system according to the present invention.

FIG. 51B is a schematic view of a system According to the present invention.

FIG. 52 is a schematic view of a system according to the present invention.

FIG. 53A is an end perspective view of a window unit according to the present invention with a pane with a coating according to the present invention.

FIG. 53B is a perspective view of a pane of the window unit of FIG. 53A.

FIG. 54 is a perspective view of a window unit according to the present invention.

FIG. 54A is a top schematic view of a pane according to the present invention.

FIG. 55A is a perspective view of a window unit according to the present invention.

FIG. 55B is a crosssection view along line 55B-55B of FIG. 55A.

FIG. 56 is a crosssection view of a window unit according to the present invention.

FIG. 57 is a crosssection view of an installation in pipe of a repair structure according to the present invention.

FIG. 58A is a perspective view of a repair structure according to the present invention.

FIG. 58B is a perspective view of a repair structure according to the present invention

FIG. 59 is a crosssection view of a repair structure according to the present invention.

FIG. 60 is a crosssection view of a pipe with a repair structure according to the present invention.

FIG. 61 is a crosssection view of a tubular according to the present invention with a repair structure according to the present invention.

FIG. 62A is a crosssection view of a joint of tubulars according to the present invention with a coupling according to the present invention.

FIG. 62B is a crosssection view of a coupling according to the present invention.

FIG. 63A is a crosssection view of a tubular (partial) according to the present invention.

FIG. 63B is a crosssection view of a tubular (partial) according to the present invention.

FIG. 64 is a crosssection view of a tubular according to the present invention with a repair structure according to the present invention.

Certain embodiments of the invention are shown in the above-identified figures and described in detail below. Various aspects and features of embodiments of the invention are described below and some are set out in the dependent claims. Any combination of aspects and/or features described below or shown in the dependent claims can be used except where such aspects and/or features are mutually exclusive. It should be understood that the appended drawings and description herein are of certain embodiments and are not intended to limit the invention or the appended claims. On the contrary, the intention is to cover all modifications, equivalents and alternatives falling within the spirit and scope of the invention as defined by the appended claims. In showing and describing these embodiments, like or identical reference numerals are used to identify common or similar elements. The figures are not necessarily to scale and certain features and certain views of the figures may be shown exaggerated in scale or in schematic in the interest of clarity and conciseness. As used herein and throughout all the various portions (and headings) of this patent, the terms “invention”, “present invention” and variations thereof mean one or more embodiments, and are not intended to mean the claimed invention of any particular appended claim(s) or all of the appended claims. Accordingly, the subject or topic of each such reference is not automatically or necessarily part of, or required by, any particular claim(s) merely because of such reference. So long as they are not mutually exclusive or contradictory any aspect or feature or combination of aspects or features of any embodiment disclosed herein may be used in any other embodiment disclosed herein. The drawing figures present the embodiments preferred at the time of filing for this patent.

### DETAILED DESCRIPTION OF THE INVENTION

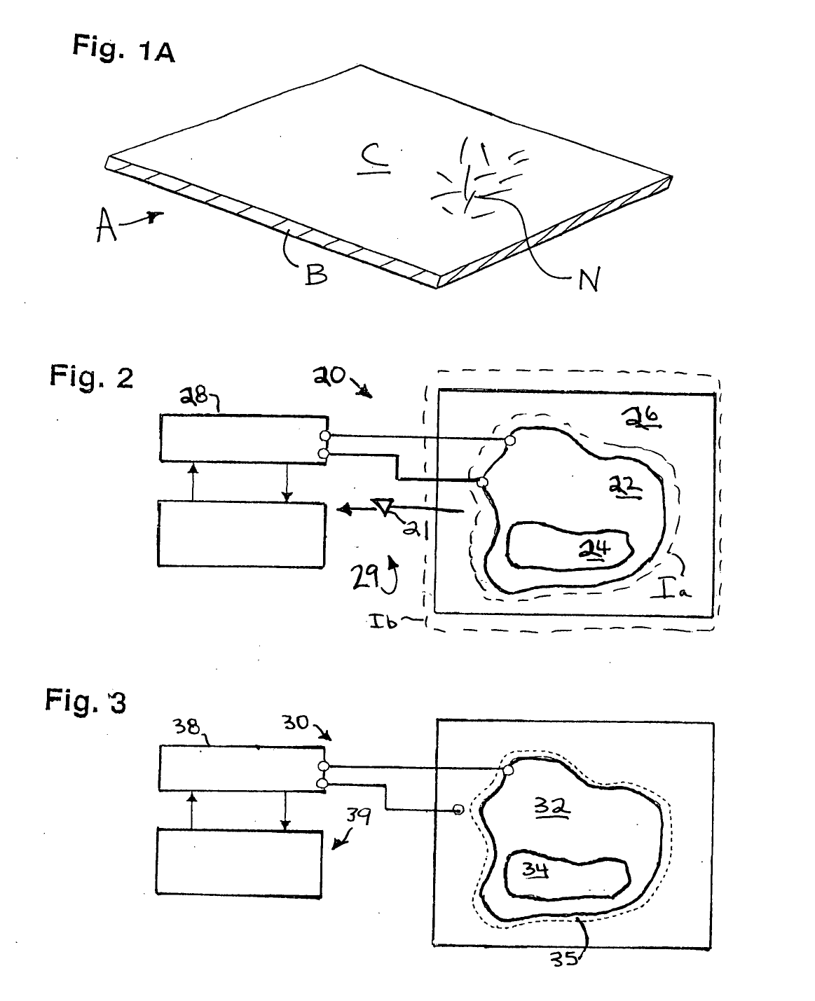

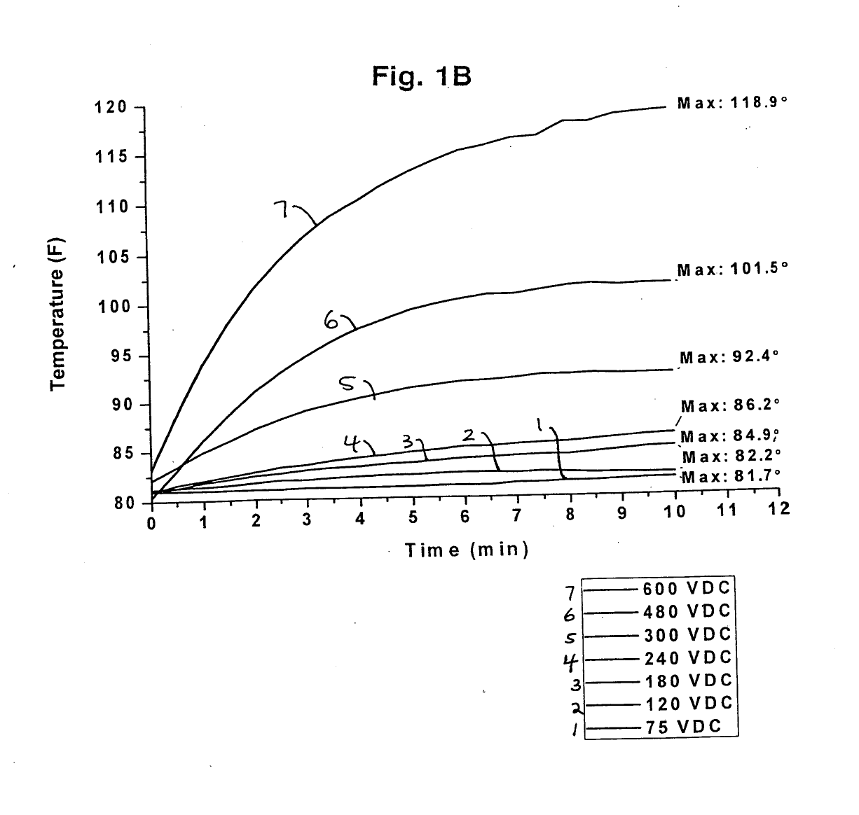

FIG. 1A shows a coating A according to the present invention (not to scale) which has a base B (e.g., 1.00 to 0.04 inches thick). Onto the base B is applied a conductive coating C with conductive nanomaterial N therein (e.g in a thin layer; e.g. between 0.005 inches thick to 0.030 inches thick). The base B may be any suitable item, surface, backing, or thing; and the conductive coating C may be any coating described or referred to herein. The nanomaterial N dispersed throughout (material N shown greatly exaggerated in size) may be any suitable nanomaterial whose use results in the effective heating of the coating; including, but not limited to, nanotubes and nanoribbons, and, in one aspect, carbon nanotubes and/or carbon nanoribbons. It is within the scope of the present invention for the base B and the coating C to be any desired thickness. In certain particular aspects, the base B is 0.06 inches thick and the coating C is between 0.010 and 0.020 inches thick.

FIG. 1B presents data for a coating according to the present invention to which seven different voltages were applied. The graph shows that as the voltage is increased (seven lines, one for each specific applied voltage, lines numbered 1-7), the temperature of the coating increases. Also, with a particular applied voltage, over time (x axis), the temperature (y axis) increases. The box with line labels 1-7 indicates the different applied voltages, from 75 VDC for line 1 to 600 VDC for line 7.

The coating of FIG. 1B includes two resins, an aliphatic polyisocyanate and a saturated polyester. These are commercially available. Fluorinated polyurethane was added (this is optional) which imparts characteristics such as low surface energy and chemical resistance for corrosive environments. This resin system (polyisocyanate, polyester, fluorinated polyurethane) was cured using an optional dibutyl tin catalyst additive. The nanomaterial used was multi-walled carbon nanotubes approximately 10 nm in diameter and with lengths ranging between 1 and 10 microns to achieve an electrical conductivity that enabled resistive heating of the coating. By weight percent, the coating components were:

1. 37% Polyisocyanate

2. 50% Polyester

3. 10% Fluorinated Polyurethane

4. 0.4% Dibutyl tin catalyst

5. 4% SMW grade Multi-walled nanotubes

The components were pre-mixed using sonication and then the resulting coating was diluted with a low-boiling-point solvent, methyl ethyl ketone (MEK), and sprayed onto the base. A high-shear homogenization mixing method may also be used (as is true with any embodiment herein). Although copper strip electrical contacts were used, any suitable electrical contacts (or leads) may be used, and they may be in any configuration, pattern and location on an item, thing, base or substrate, with any suitable spacing between them; e.g., but not limited to, in parallel, in a spiral configuration, or in a helical configuration.

FIG. 2 shows a system 20 according to the present invention which includes a heatable coating 22 which, in one aspect, is a deicing coating 22 according to the present invention (any coating according to the present invention disclosed herein) to affect ice 24 that might adhere to an item 26. The item 26 may be any item or surface subjected to icing (including, but not limited to, any item substrate, or surface disclosed herein); for example, and not by way of limitation, the item 26 may be an airplane wing, a helicopter blade, a turbine blade, a jet inlet, a heat exchanger for kitchen and industrial equipment, a refrigerator, a road sign, machines, oil filed apparatuses, oil field devices, oil field structures, a ship hull or superstructures, any item or surface disclosed herein, or other object or surface subjected to adverse, cold, wet and/or ice conditions. More specifically, the coating 22 is applied over the item 26 to protect it, e.g., but not limited to, to protect it from ice 24. The coating 22, in one aspect, is either sprayed-on or is flexible prior to application so that it physically conforms to the shape of item 26. In operation, a voltage is applied to the coating 22 by a power supply 28 (AC or DC). In certain aspects, this voltage is between 2 volts and 1000 volts and, in one aspect, with higher voltages being applied for lower temperatures. Whether the coating 22 is a protective coating, a heatable coating, or a deicing coating according to the present invention, the electrical system 20 may be used with a sensor 21 which senses an event and/or a parameter (of the coating, of the item, and/or of the environment) related to the item with the coating. For example, a sensed event may be the formation of ice, a change in the coating, or a change in temperature. Upon such sensing occurring, the electrical system can heat the coating, e.g. to change the temperature or to deice the item.

When voltage is applied, the ice 24 is heated and then melts to water and flows from the surface. Further, gases from within the ice 24 and generate pressure bubbles that exfoliate ice 24 from the coating 22 (and hence from the item 26). Optionally, a voltage regulator subsystem 29 (optionally with a sensor, temperature sensor or sensors) is connected in feedback with the power supply 28, and thereby with the circuit formed by the coating 22 and the ice 24. The subsystem 29 increases or decreases DC voltage applied to the coating 22 as required. Optionally, the coating 22 and/or the item 26 has an insulator thereon to facilitate the maintenance of a desired current flow through the coating and/or so that relatively less current is needed to maintain a desired current flow; e.g., an insulating coating, blanket, covering or sleeve Ia on the coating 22 or an insulating coating, blanket, covering or sleeve Ib on the item 26—each shown in dotted lines.

FIG. 3 shows a system 30 according to the present invention which has an electrical deicing coating 32 according to the present invention to affect ice 34 that might adhere to a conductive surface 36. The conductive surface 36 may be on any item subjected to icing. The coating 32 is applied over the surface 36 to protect the surface 36 from the ice 34. In one aspect, the coating 32 is flexible so as to physically conform to the shape of surface 36. In operation, a voltage is applied between the coating 32 and the surface 36 by a power supply 38. A bias voltage applied to the coating 32 may be equal and opposite to a bias voltage applied to the surface 36. If desired, an insulator 35 may be disposed between the coating 32 and the surface 36. The insulator 35 may be any known suitable insulator, insulating structure, or insulating material. Optionally, an insulator like either or both of the insulators Ia and Ib, FIG. 2, is used.

If desired, a voltage regulator subsystem 39 (e.g. the one in FIG. 2) is connected in feedback with the power supply 38, and thereby with the circuit formed by the coating 32, the surface 36, and the ice 34, so as to increase or decrease the DC voltage applied to the coating 32.

In the embodiments that follow, any item, thing, or surface may be coated with any coating according to the present invention and any system according to the present invention may be used to apply a voltage to the coating, including, but not limited to, the coatings and systems described above in FIGS. 1-3.

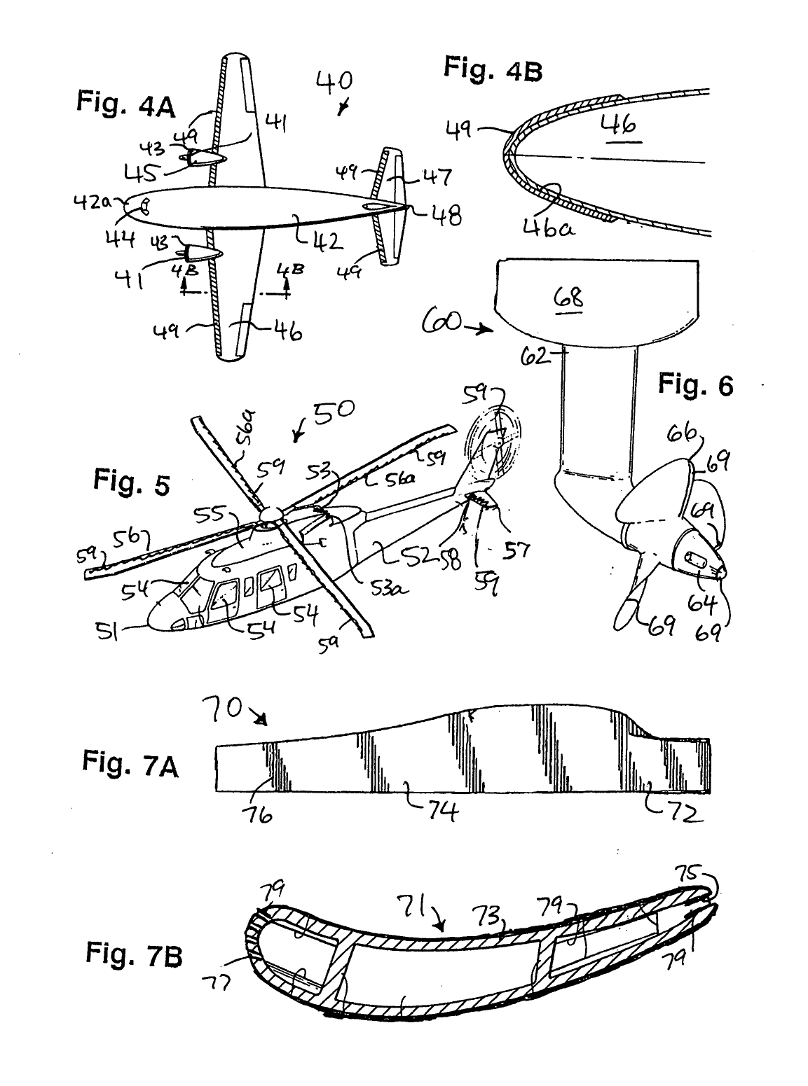

FIG. 4A shows a plane 40 with a body 42, a nose 42a, window 44, wings 46, engines 45, and tail 48. It is within the scope of the present invention to provide a coating according to the present invention and/or coating system according to the present invention to any part of a plane, including, and not limited to, the body, windows, wings, wing leading edge, engine inlets, engine housings, and tail, as well as any particular part, projection, or instrument which may be subjected to icing. Such parts, etc., may be entirely covered with the coating or only part thereof may be covered with the coating.

As shown in FIG. 4B, a leading edge 46a of the wings and a horizontal tail portion 47 are coated with a coating 49 according to the present invention and a system 40s (like any system disclosed herein) provides the required voltage to heat the coating. Engine inlets 43 (which engines may be any kinds of engine; e.g., jet, turbine, piston, rocket, etc.) are coated with a coating 41 according to the present invention.

FIG. 5 shows a helicopter 50 with a body 52, a nose 51, windows 54, rotors 56, engine 55, tail 58, and tail propeller 59. It is within the scope of the present invention to provide a coating according to the present invention and/or coating system according to the present invention to any part of a helicopter, including, and not limited to, the body, windows, rotors, engine inlet, engine housing, tail, and tail propeller, as well as any particular part, projection, or instrument which may be subjected to icing. Such parts, etc., may be entirely covered with the coating or only part thereof may be covered with the coating.

As shown in FIG. 5, a leading edge 56a of the rotors and of a horizontal tail portion 57 are coated with a coating 59 according to the present invention and a system 50s (like any system disclosed herein) provides the required voltage to heat the coating. An engine inlet 53 (which engines may be any kinds of engine; e.g., jet) is coated with a coating 53a according to the present invention.

FIG. 6 shows a boat motor 60 with a body 62, a nose 64, a propeller 66, and a motor housing 68. It is within the scope of the present invention to provide a coating according to the present invention and/or coating system according to the present invention to any part of a boat motor, including, and not limited to, the body, nose, propeller, motor housing, and propeller, as well as any particular part, projection, or instrument which may be subjected to icing. Such parts, etc., may be entirely covered with the coating or only part thereof any be covered with the coating. Edges of the propeller 66 and the tip of the nose 64 are coated with a coating 69 according to the present invention and a system 60s is used to apply a voltage to the coating areas.

FIG. 7A shows a turbine blade 70 with a body 72 and vane portion 74. A coating 76 according to the present invention (shown partially in exaggerated size) coats the body and the vane portion. A control system 70s applies a voltage to the coating 76.

FIG. 7B shows a turbine blade 71 with a body 73, an inlet 75, and inlets 77. A coating 79 according to the present invention (shown partially in exaggerated size) coats the body and the inlets. A control system 71s applies a voltage to the coating 79.

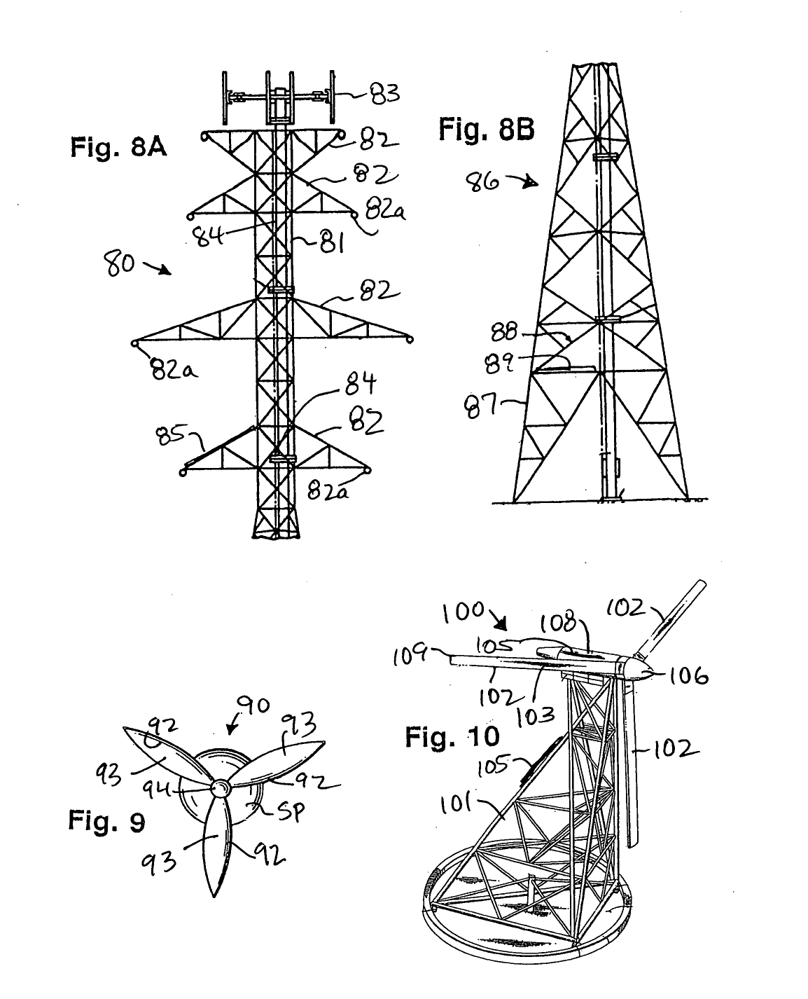

FIG. 8A shows a power transmission tower 80 with a main structure 81, arms 82, insulators 82a, top structure 83, and interior structure 84. These parts are coated with a coating 85 according to the present invention (shown partially in exaggerated size on one of the arms 82) and a system 80s provides the voltage to be applied to the coating. It is within the scope of the present invention to delete the coating from any part of the tower 80 or to coat only one of the parts.

FIG. 8B shows a power transmission tower 86 with a main structure 87 and interior structure 88. These parts are coated with a coating 89 according to the present invention (shown partially in exaggerated size on part of the interior structure 89) and a system 86s provides the voltage to be applied to the coating. It is within the scope of the present invention to delete the coating from any part of the tower 86 or to coat only one of the parts.

FIG. 9 shows a propeller 90 according to the present invention with a coating 92 on blades 93 and on a hub 94 mounted to a support SP. A system 90s according to the present invention provides the voltage to the coating 92 to heat the coating. It is within the scope of the present invention to delete the coating from any part of the propeller 90, to coat an edge of the blades 93, to coat a leading edge of the blades 93, or to coat one of the parts of the propeller, or only one of these parts.

FIG. 10 shows a wind power generating system 100 according to the present invention with a rotatable propeller on a support 101. The propeller 109 has blades 102 and a nose 106. A power conversion system 108 converts the rotative force of the propeller into usable power. The blades 102 and/or the nose 106 are coated with a coating 103 according to the present invention (shown in exaggerated size) and a system 100s provides the voltage to heat the coating 103. Optionally, there is a coating 105 according to the present invention (shown partially in exaggerated size) on other parts of the system 100. It is within the scope of the present invention to delete the coating from any part or parts of the system 100, to coat an edge of the blades 103, to coat a leading edge of the blades 103, to coat the nose 106, and/or to coat one of the parts of the propeller, or only one of these parts.

FIG. 11A shows a pipeline 110 according to the present invention on supports ST above the earth ER with a coating 112 according to the present invention (shown partially in exaggerated size) on its exterior. A system 110s according to the present invention provides the voltage to the coating 112 to heat the coating. It is within the scope of the present invention to delete the coating from any part of the pipeline 110, to coat the bottom half of the pipeline, to coat substantially all of the exterior surface of the pipeline, or to coat a bottom strip along the entire pipeline or only along certain parts of the pipeline, or only one of these parts.

FIG. 11B shows a pipeline 114 according to the present invention within the earth ET with a coating 116 according to the present invention (shown partially in exaggerated size) on its exterior. A system 114s according to the present invention provides the voltage to the coating 116 to heat the coating. It is within the scope of the present invention to delete the coating from any part of the pipeline 114, to coat the bottom half of the pipeline, to coat substantially all of the exterior surface of the pipeline, or to coat a bottom strip along the entire pipeline or only along certain parts of the pipeline, or only one of these parts.

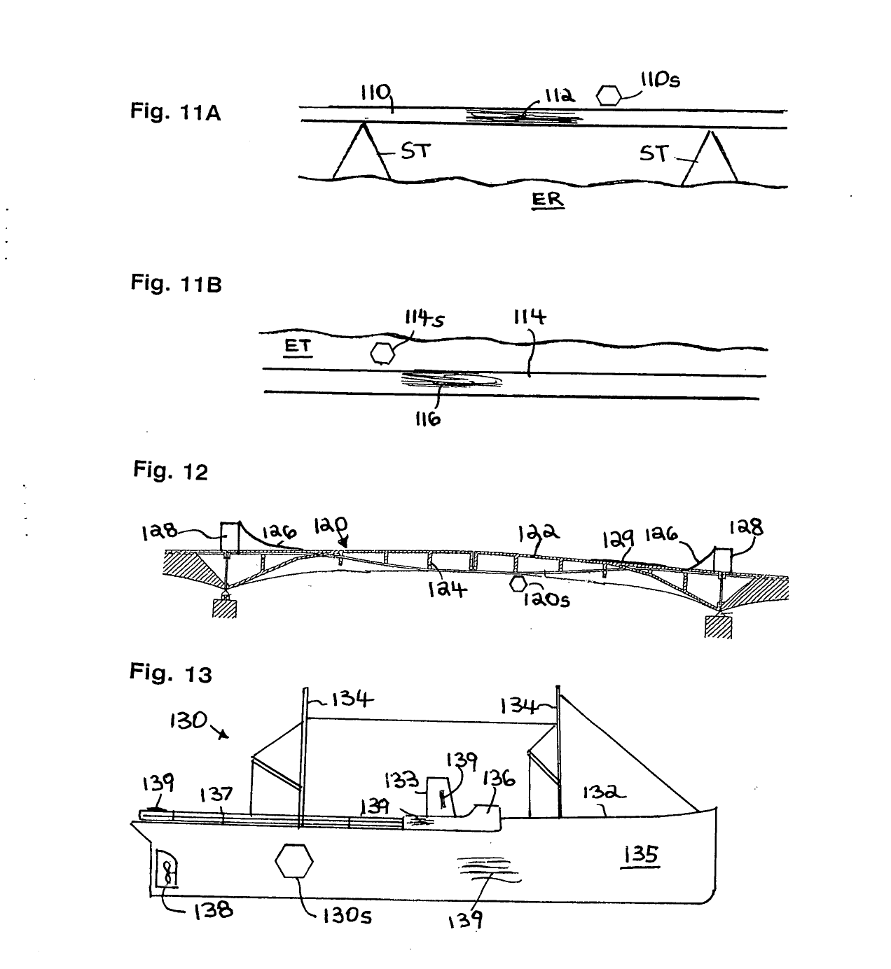

FIG. 12 shows a bridge 120 according to the present invention with a deck 122, supporting structure 124, cables 126, and towers 128. A coating 129 according to the present invention (shown partially in exaggerated size) coats exterior surfaces of the parts of the bridge 120. A system 120s according to the present invention provides the voltage to the coating 129 to heat the coating. It is within the scope of the present invention to delete the coating from any part of the bridge 120, to coat the deck of the bridge, to coat the cables, and/or to coat the support structure, or only one of these parts.

FIG. 13 shows a ship 130 according to the present invention with a deck 132, superstructure 133, masts 134, hull 135, cabin 136, railing 137, and drive propeller 138. A coating 139 according to the present invention (shown partially in exaggerated size) coats exterior surfaces of the parts of the ship 130. A system 130s according to the present invention provides the voltage to the coating 139 to heat the coating. It is within the scope of the present invention to delete the coating from any part of the ship 130, to coat the deck, the hull, the superstructure, the cabin, the railing and/or the masts, the bow of the ship, and/or to coat the bottom of the hull, or only one of these parts. Any coating herein may be used on any suitable part of a cabin or other building, whether or not it is one a ship, including permanent structures and portable buildings (and including tents); including, but not limited to, walls, doors, tent flaps, tent walls, tent floors, and windows of such structures, buildings, and tents.

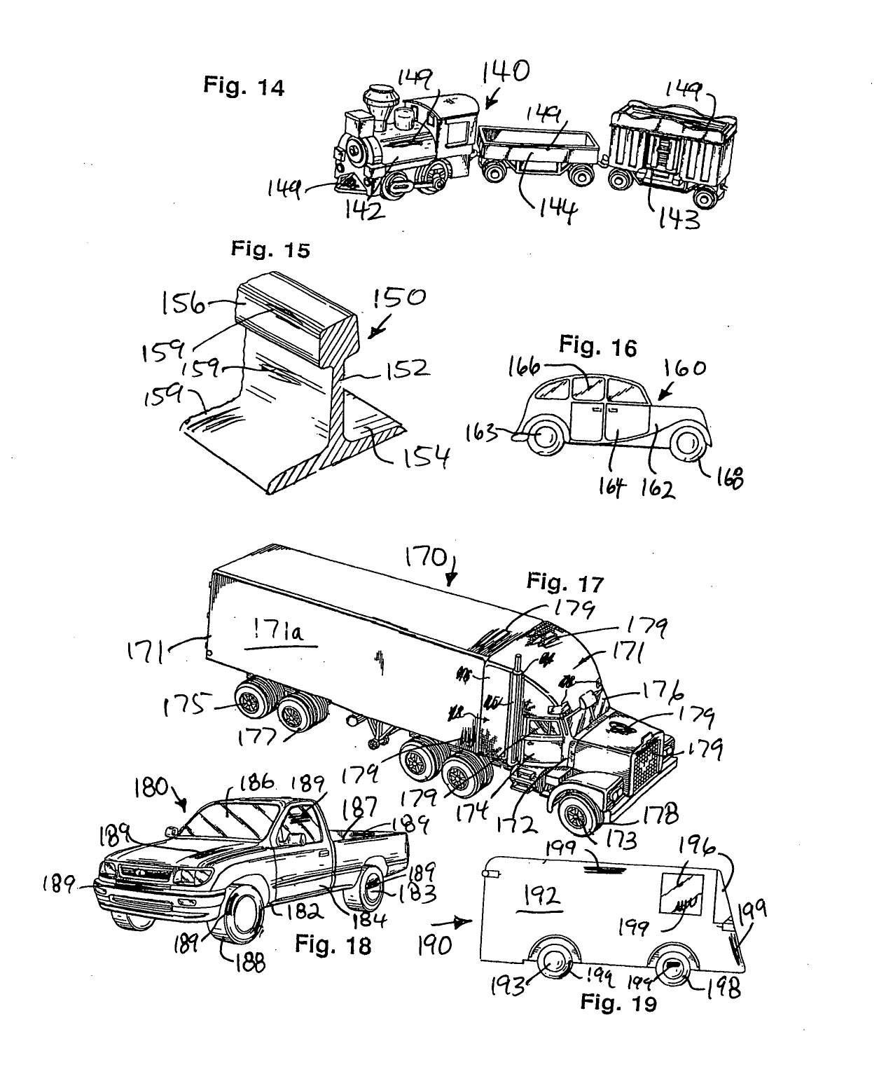

FIG. 14 shows a train 140 according to the present invention with a locomotive 142, car 143, and car 144. A coating 149 according to the present invention (shown partially in exaggerated size) coats exterior surfaces of the parts of the train 140. A system 140s according to the present invention provides the voltage to the coating 149 to heat the coating. It is within the scope of the present invention to delete the coating from any part of the train 140, to coat only the front of the locomotive, the tops of the locomotive and/or car(s), and/or a part or parts of the locomotive and/or cars, or only one of these parts.

FIG. 15 shows a rail 150 according to the present invention with a body 152, a base 154, and a top 156. A coating 159 according to the present invention (shown partially in exaggerated size) coats exterior surfaces of the parts of the rail 150. A system 150s according to the present invention provides the voltage to the coating 159 to heat the coating. It is within the scope of the present invention to delete the coating from any part of the rail 150, to coat the base, the body, the top of the top, and/or the top of the rail, or only one of these parts.

FIG. 16 shows an automobile 160 according to the present invention with a body 162, doors 164, windows 166, tires 168, and wheels 163. A coating 169 according to the present invention (shown partially in exaggerated size) coats exterior surfaces of the parts of the automobile 160. A system 160s according to the present invention provides the voltage to the coating 169 to heat the coating. It is within the scope of the present invention to delete the coating from any part of the automobile 160, from the tires, the body, the top of the automobile, the front of the automobile, the wheels, the top of the top, the top of the hood, and/or the windows, or only one of these parts.

FIG. 17 shows a tractor trailer rig 170 according to the present invention with a tractor 171 with a body 172, doors 174, windows 176, tires 178, and wheels 173; and with a trailer 171 with a body 171a, wheels 175, and tires 177. A coating 179 according to the present invention (shown partially in exaggerated size) coats exterior surfaces of the parts of the automobile tractor trailer rig 170. A system 170s according to the present invention provides the voltage to the coating 179 to heat the coating. It is within the scope of the present invention to delete the coating from any part of the tractor trailer rig 170, to coat the tires, the bodies, the top of the bodies, the front of the tractor, the wheels, only the top of the hood of the tractor, and/or the windows, or only one of these parts.

FIG. 18 shows a pick-up truck 180 according to the present invention with a body 182, doors 184, windows 186, tires 188, a bed 187, and wheels 183. A coating 189 according to the present invention (shown partially in exaggerated size) coats exterior surfaces of the parts of the truck 180. A system 180s according to the present invention provides the voltage to the coating 189 to heat the coating. It is within the scope of the present invention to delete the coating from any part of the truck 180, to coat the tires, the body, the top of the vehicle, the front of the vehicle, the wheels, the top of the top, and/or the windows, or only one of these parts.

FIG. 19 shows a recreational vehicle 190 according to the present invention with a body 192, windows 196, tires 198, and wheels 193. A coating 199 according to the present invention (shown partially in exaggerated size) coats exterior surfaces of the parts of the vehicle 190. A system 190s according to the present invention provides the voltage to the coating 199 to heat the coating. It is within the scope of the present invention to delete the coating from any part of the vehicle 190, to coat the tires, only the body, the top of the vehicle, the front of the vehicle, the wheels, and/or the windows or only one of these parts.

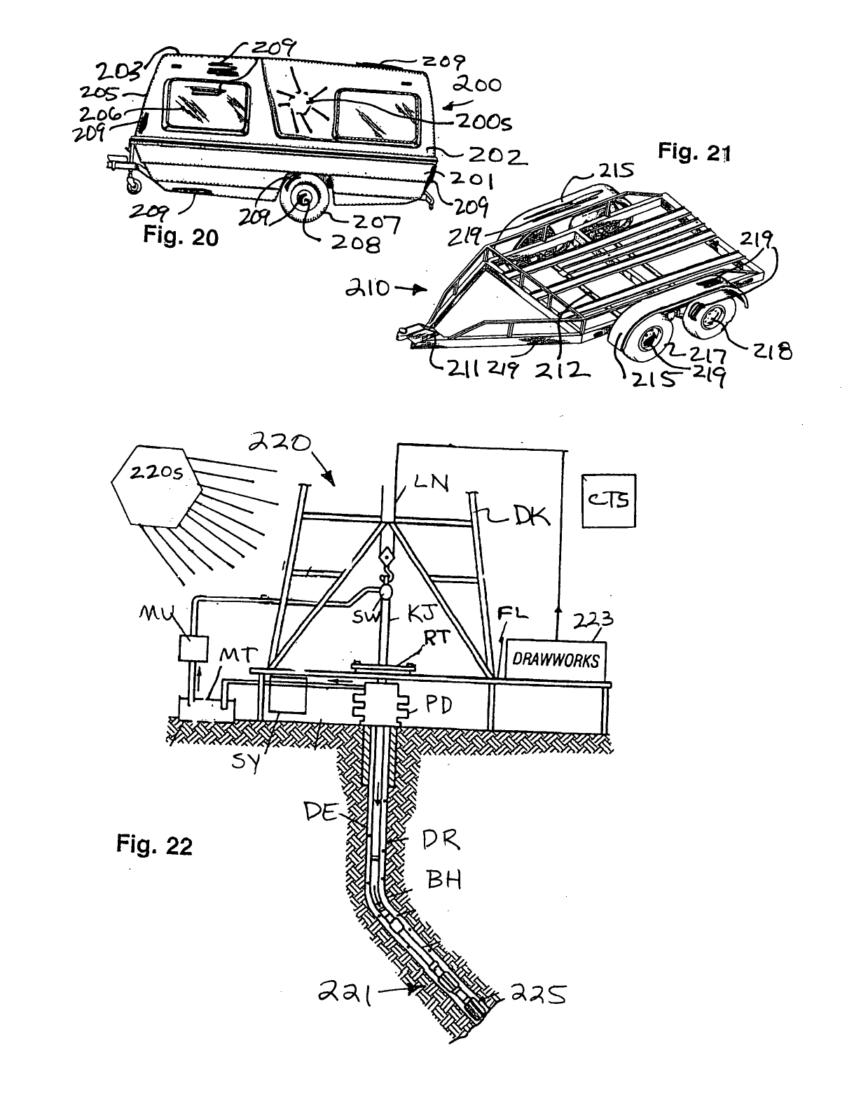

FIG. 20 shows a travel trailer 200 according to the present invention with a body 202, rear 201, roof 203, bottom 204, front 205, windows 206, tires 207, and wheels 208. A coating 209 according to the present invention (shown partially in exaggerated size) coats exterior surfaces of the parts of the trailer 200. A system 200s according to the present invention (shown schematically as are the similar systems in the figures described above) provides the voltage to the coating 209 to heat the coating. It is within the scope of the present invention to delete the coating from any part of the trailer 200, to coat the tires, the body, the top of the vehicle, the front or bottom or rear of the vehicle, the wheels, and/or the windows or only one of these parts.

FIG. 21 shows a utility trailer 210 according to the present invention with a body 212, tongue 211, fenders 215, tires 217, and wheels 218. A coating 219 according to the present invention (shown partially in exaggerated size) coats exterior surfaces of the parts of the trailer 210. A system 200s according to the present invention (shown schematically as are the similar systems in the figures described above) provides the voltage to the coating 209 to heat the coating. It is within the scope of the present invention to delete the coating from any part of the trailer 210, to coat the tires, the body, the tongue, the fenders, the wheels, the front or bottom or rear of the trailer, or only one of these parts. As is true for any embodiment herein which has an electrical system designated with a numeral followed by a lower case “s,” the system 220s may be any suitable voltage applications system, apparatus, or device, including, but not limited to, those disclosed or referred to herein.

FIG. 22 shows a schematic diagram of an exemplary drilling system 220 according to the present invention having a drilling assembly 221 shown conveyed in a borehole BH for drilling a wellbore. The drilling system 220 includes a conventional derrick DK having a floor FL which supports a typical rotary table RT that is rotated by a prime mover whose motor (not shown) is controlled by a motor controller (not shown). It is within the scope of the present invention to use the teachings of the present invention for known drilling methods and techniques, including, but not limited to, rotary drilling, top drive drilling, casing drilling, and coil tubing drilling. In one aspect, a drillstring DR includes a drill pipe DE extending downward from the rotary table through a pressure control device PD (e.g., but not limited to, one or more BOP's) into the borehole. A drill bit 225, attached to the drillstring end, disintegrates the geological formations when it is rotated to drill the borehole. The drill string is coupled to a drawworks 223 via a kelly joint KJ, swivel SW and line LN through a pulley (not shown). This description is drawn to a land rig, but the invention as disclosed herein is also equally applicable to any offshore drilling rigs or systems. Alternatives to conventional drilling rigs, such as coiled tubing systems (shown schematically as CTS), can be used to drill boreholes, and the invention disclosed herein is equally applicable to such systems. Mud pump MU pumps drilling fluid into the drill string via the kelly joint KJ and the drilling fluid is discharged at the borehole bottom through an opening in the drill bit. The drilling fluid circulates uphole through an annular space between the drill string and the borehole and returns to a mud tank MT via a solids control system SY. The solids control system may include shale shakers, centrifuges, and other known solids control equipment.

In one aspect, each, some, or substantially all of the exterior surfaces of the equipment, devices, conduits, and items described above for the system 220 is/are coated with a coating 229 according to the present invention (shown on some parts exaggerated in size) and a system 220s according to the present invention (shown schematically) provides the voltage to heat the coating. It is within the scope of the present invention to so coat only one of the pieces of equipment, etc. of the system 220 according to the present invention.

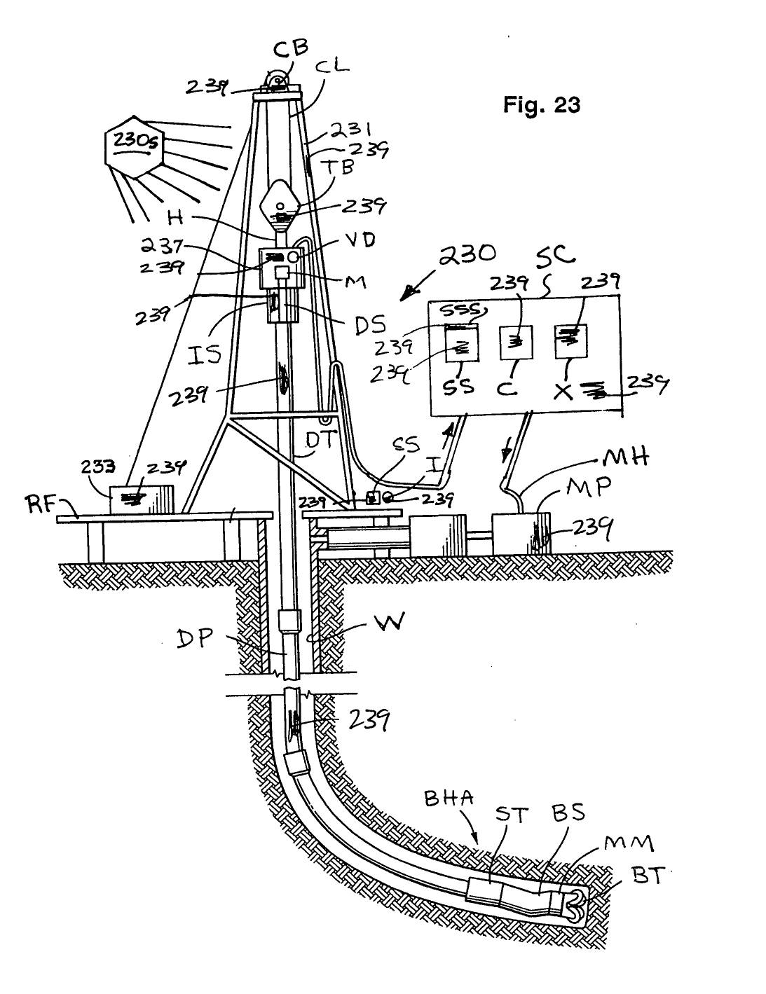

Referring now to FIG. 23, a drilling rig 230 according to the present invention is depicted schematically as a land rig, but other rigs (e.g., offshore rigs, jack-up rigs, semisubmersibles, drill ships, and the like) are within the scope of the present invention (and this is true for the embodiments of rigs and wellbore operations described below also). In conjunction with an operator interface, e.g. an interface I, a control system CS controls certain operations of the rig. The rig 230 includes a derrick 231 that is supported on the ground above a rig floor RF. The rig 230 includes lifting gear, which includes a crown block CB mounted to the derrick 231 and a traveling block TB. The crown block and the traveling block are interconnected by a cable CL that is driven by drawworks 233 to control the upward and downward movement of the traveling block. The traveling block carries a hook H from which is suspended a top drive system 237 which includes a variable frequency drive controller VD, a motor M (or motors) and a drive shaft DS. The top drive system 237 rotates a drillstring DT to which the drive shaft is connected in a wellbore W. The drillstring is coupled to the top drive system through an instrumented sub IS which can include sensors that provide information, e.g., drillstring torque information. The drillstring may be any typical drillstring and, in one aspect, includes a plurality of interconnected sections of drill pipe DP a bottom hole assembly BHA, which includes appropriate stabilizers, drill collars, and/or an apparatus or device, in one aspect, a suite of measurement while drilling (MWD) instruments including a steering tool ST to provide bit face angle information. Optionally a bent sub BS is used with a downhole or mud motor MM and a bit BT, connected to the BHA. Drilling fluid is delivered to the drillstring by mud pumps MP through a mud hose MH. During rotary drilling, the drillstring is rotated within the bore hole by the top drive system. Fluid from the well and the cuttings produced as the bit drills into the earth are carried out of bore hole by the fluid supplied by the mud pumps. The fluid expelled from the well flows to solids control equipment SC which may include one or more shale shakers SS with one or more shale shaker screens SSS; one or more centrifuges C; and/or other fluid processing equipment X (e.g., but not limited to, degassers, desilters, desanders, and hydrocyclones).

In one aspect, each, some, or substantially all of the exterior surfaces of the equipment, devices, conduits, and items described above for the rig 230 is/are coated with a coating 239 according to the present invention (shown on some parts exaggerated in size) and a system 230s according to the present invention (shown schematically) provides the voltage to heat the coating. It is within the scope of the present invention to so coat only one of the pieces of equipment, etc. of the rig 230 according to the present invention. It is also within the scope of the present invention (as is true for any part of any embodiment herein) to coat a part to heat it, for heating and/or for deicing; e.g., for heating to facilitate flow, to remediate wax build-up in a conduit or pipeline, to inhibit or reduce hydrate formation in a conduit or pipeline, to deice a well or wellbore which is cased or uncased, to control viscosity of the contents of a storage tank, to provide a conductive path or liner for a storage tank, and to improve gas permeation of a fabric.

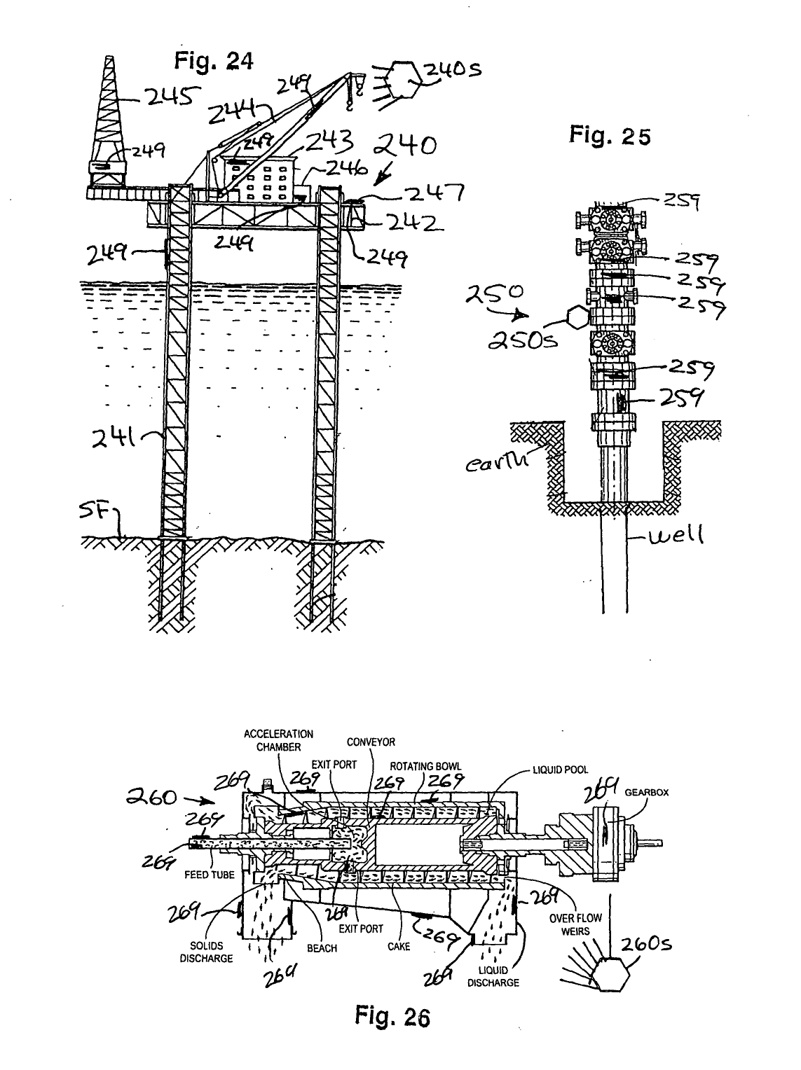

FIG. 24 shows an offshore rig 240 according to the present invention which has legs 241 that extend down to and beneath a seafloor SF. The legs 241 support a platform 242 that has a cabin 243 (or portable building), a lifting apparatus 244, a derrick 245, a deck 247, and the equipment, devices, conduits, items, and apparatuses (shown schematically and all designated by the box labeled 246) necessary for operations on the rig 240.

According to the present invention, one, some, or substantially all of the equipment, devices, conduits, apparatuses, and items of the rig 240 are coated with a coating 249 according to the present invention (shown on some parts in exaggerated size) and a system 240s provides the voltage for heating the coating. In certain aspects according to the present invention, only one item, etc., or only one part, etc. has this coating; and it is within the scope of the present invention to delete the coating from any part of the rig 240.

FIG. 25 shows a blowout preventer system 250 according to the present invention. According to the present invention, one, some, or substantially all of the equipment, devices, conduits, apparatuses, parts, and items of the BOP system 250 are coated with a coating 259 according to the present invention (shown on some parts in exaggerated size). A system 250s according to the present invention provides the voltage to the coating 259 to heat the coating. It is within the scope of the present invention to delete the coating from any part of the system 250 or to coat only one of these parts.

FIG. 26 shows a centrifuge 260 according to the present invention which has the parts as labeled in FIG. 26. According to the present invention, one, some, or substantially all of the equipment, devices, conduits, apparatuses, parts, and items of the centrifuge 260 are coated with a coating 269 according to the present invention (shown on some parts in exaggerated size). A system 260s according to the present invention provides the voltage to the coating 269 to heat the coating. It is within the scope of the present invention to delete the coating from any part of the centrifuge 260 or to coat only one of these parts.

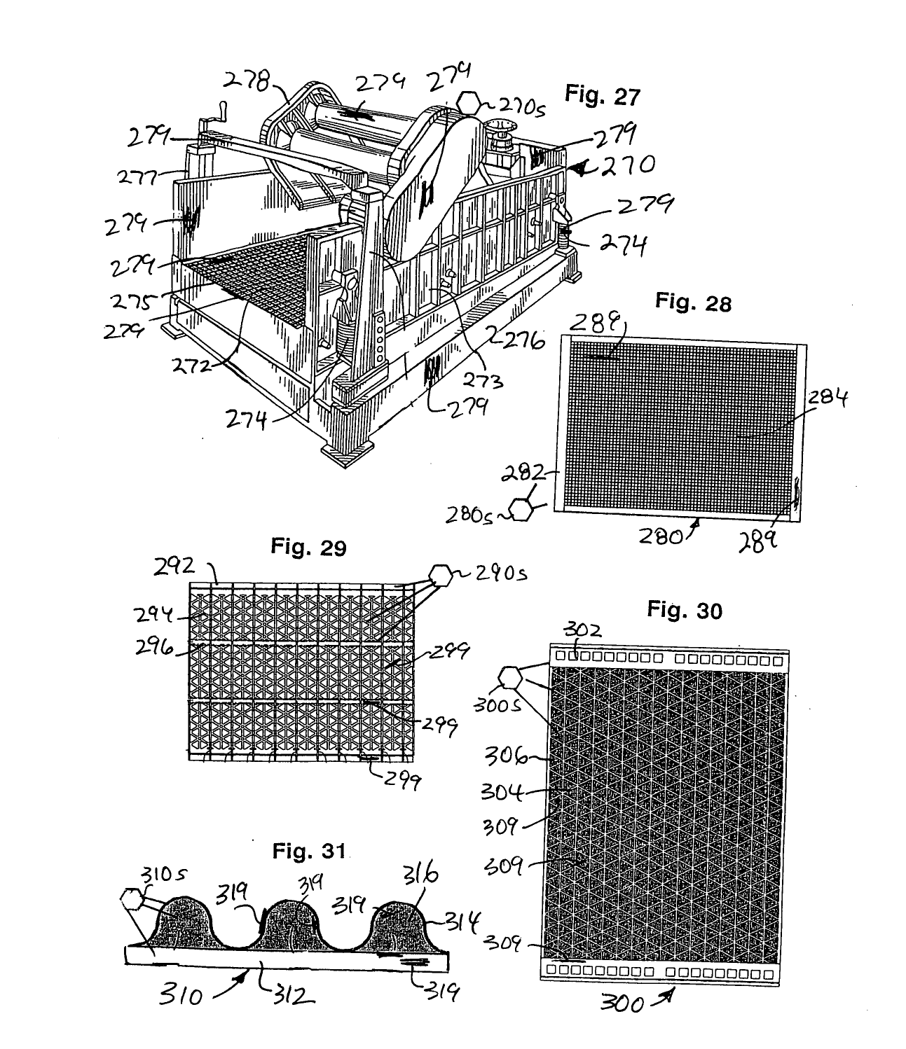

FIG. 27 shows a shale shaker 270 according to the present invention which has a screen 272 (or screens) (with screen or screening cloth or mesh as desired) mounted on vibratable screen mounting apparatus or “basket” 273 with a screen support 275. The screen(s) 272 may be any known screen or screens, but with a coating on some or all parts according to the present invention. The basket 273 is mounted on springs 274 which are supported from a frame 276. The basket 273 is vibrated a vibrating apparatus 278 which is mounted on the basket 273 for vibrating the basket and the screens. Elevator apparatus 277 provides for raising and lowering of the basket end. According to the present invention, one, some, or substantially all of the equipment, devices, conduits, apparatuses, parts, and items of the shaker 270 are coated with a coating 279 according to the present invention (shown on some parts in exaggerated size). A system 270s according to the present invention provides the voltage to the coating 279 to heat the coating. It is within the scope of the present invention to delete the coating from any part of the shaker 270 or to coat only one of these parts.

FIG. 28 shows a shale shaker screen 280 according to the present invention which has a frame 282 and screen mesh 284. According to the present invention, the frame (top, sides and/or bottom) is coated with a coating 289 according to the present invention (shown on some parts in exaggerated size); and/or the screen mesh (top and/or bottom) is so coated. A system 280s according to the present invention provides the voltage to the coating 289 to heat the coating. It is within the scope of the present invention to delete the coating from any part of the screen 280 or to coat only one of these parts.

FIG. 29 shows a shale shaker screen support 290 according to the present invention which has a sides 292, cross members 296, and mesh support grid 294. According to the present invention, sides, cross members, and/or grid are coated with a coating 299 according to the present invention (shown on some parts in exaggerated size). A system 290s according to the present invention provides the voltage to the coating 299 to heat the coating. It is within the scope of the present invention to delete the coating from any part of the screen support 290 or to coat only one of these parts.

FIG. 30 shows a shale shaker screen 300 according to the present invention which has a sides 302, screening material 306, and grid 304 (made, e.g., of metal or of epoxy). According to the present invention, the sides, screening material (top and/or bottom), and/or grid are coated with a coating 309 according to the present invention (shown on some parts in exaggerated size). A system 300s according to the present invention provides the voltage to the coating 309 to heat the coating. It is within the scope of the present invention to delete the coating from any part of the screen 300 or to coat only one of these parts.

FIG. 31 shows a shale shaker screen 310 according to the present invention which has a support 312 and three-dimensional screening material 314 (with raised portions or “hills” between lower portions or “valleys”) on the support 312. Optionally, end of the 3-D material are closed off with screening material 316 (or with a solid material). According to the present invention, the sides, screening material (top and/or bottom), and/or grid are coated with a coating 319 according to the present invention (shown on some parts in exaggerated size). A system 310s according to the present invention provides the voltage to the coating 319 to heat the coating. It is within the scope of the present invention to delete the coating from any part of the screen 310 or to coat only one of these parts.

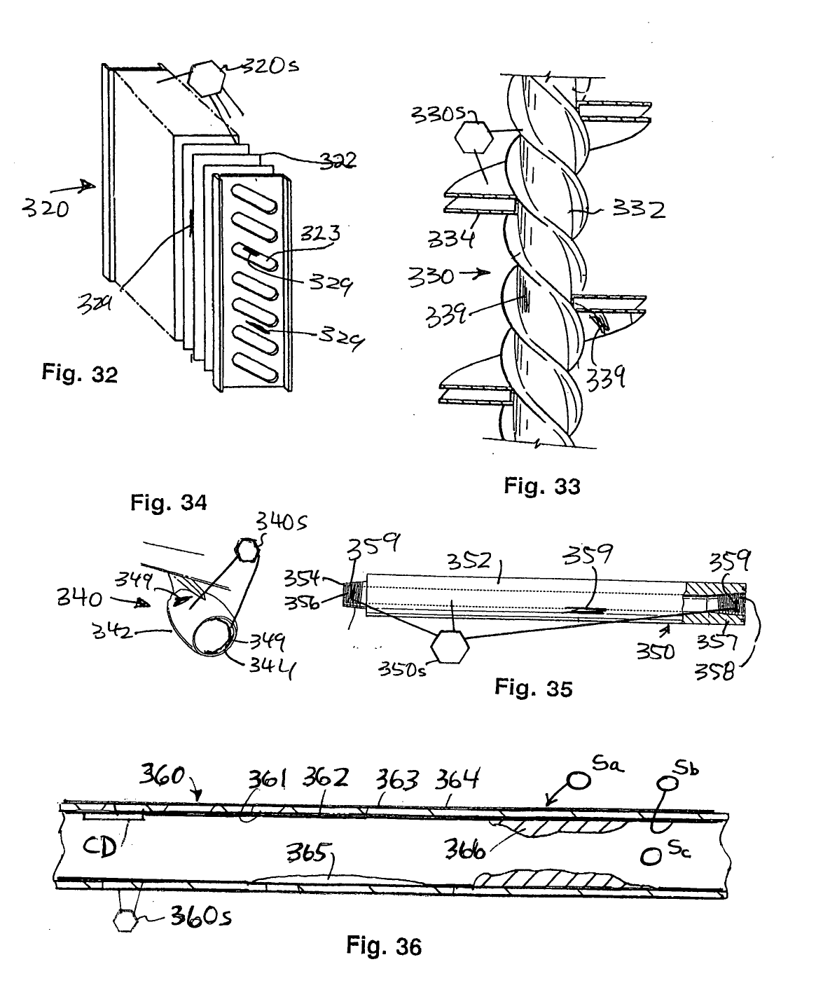

FIG. 32 shows a heat exchanger 320 according to the present invention which has fin plates 322 and tubes 323 extending through the plates 322. According to the present invention, the plates, and/or the tubes are coated with a coating 329 according to the present invention (shown on some parts in exaggerated size). A system 320s according to the present invention provides the voltage to the coating 309 to heat the coating. It is within the scope of the present invention to delete the coating from any part of the exchanger 320 or to coat only one of these parts.

FIG. 33 shows a finned tube 330 according to the present invention for a heat exchange. The tube 330 has a hollow tubular body 332 with a plurality of spaced-apart fins 334. According to the present invention, the body (inside and/or outside), and/or the fins (either or both sides) are coated with a coating 339 according to the present invention (shown on some parts in exaggerated size). A system 330s according to the present invention provides the voltage to the coating 339 to heat the coating. It is within the scope of the present invention to delete the coating from any part of the tube 330 or to coat only one of these parts.

FIG. 34 shows a jet engine 340 according to the present invention, e.g., for a plane. The engine 340 has a housing 342 with an inlet 344. According to the present invention, the housing (inside and/or outside), and/or the inlet (inside and/or outside) are coated with a coating 349 according to the present invention (shown on some parts in exaggerated size). A system 340s according to the present invention provides the voltage to the coating 349 to heat the coating. It is within the scope of the present invention to delete the coating from any part of the engine 340 or to coat only one of these parts.

FIG. 35 shows a tubular 350 according to the present invention. The tubular 350 has a channel 351 therethrough from one end to the other. Optionally, the tubular 350 has a pin end 354 with threading 356 and a box end 357 with threading 358. According to the present invention, the tubular 350 (inside and/or outside), and/or either end (inside and/or outside), and/or the pin end, and/or the box end, and/or the threading on either or both ends are coated with a coating 359 according to the present invention (shown on some parts in exaggerated size). A system 350s according to the present invention provides the voltage to the coating 359 to heat the coating. It is within the scope of the present invention to delete the coating from any part of the tubular 350 or to coat only one of its parts. In certain aspects, and not by way of limitation, the tubular is tubing, casing, pipe, drill pipe, drill collar, riser, or oil country tubular goods.

FIG. 36 shows a conduit 360 according to the present invention (which, in one aspect, is a pipeline for the transport of fluid, e.g., but not limited to, steam, gas, chemicals, oil, or hydrocarbons, or is well-known casing, tubing, pipe, or riser). The conduit 360 has an interior 361 coated with a coating 362 according to the present invention and/or an exterior 363 coated with a coating 364 according to the presenting invention. A system 360s according to the present invention provides the voltage to the coating(s) to heat the coating(s). Such heating can inhibit or reduce the formation of hydrates (and/or condensation) 365 within the conduit 360 and/or can inhibit or assist in removing deposits on the conduit, e.g., but not limited to, the treatment of paraffin wax 366 on the interior 361 of the conduit 360. The coatings are not shown to scale (as is true of all the drawings herein). Optionally, sensor systems Sa, Sb, and/or Sc monitor and/or sense any change of or parameter of the coatings. Sensor systems Sa and Sb are exterior to the conduit, with the system Sa sensing the exterior coating and sensor system Sb sensing the interior coating. The sensor system Sc is within the conduit, either attached to the conduit or not. In one aspect, the sensor system(s) sense a change in the coating; e.g., but not limited to a change in the coating as originally applied (either factory applied or on-site applied) and the coating post use, post joint makeup, post wellbore insertion, or post other treatment; e.g., but not limited to, a change related to cracking of the coating, dissolution of the coating, abrasion of the coating, or deterioration of the coating. Such sensor(s) can be used on with any other item, thing, base or substrate disclosed herein with a coating according to the present invention.

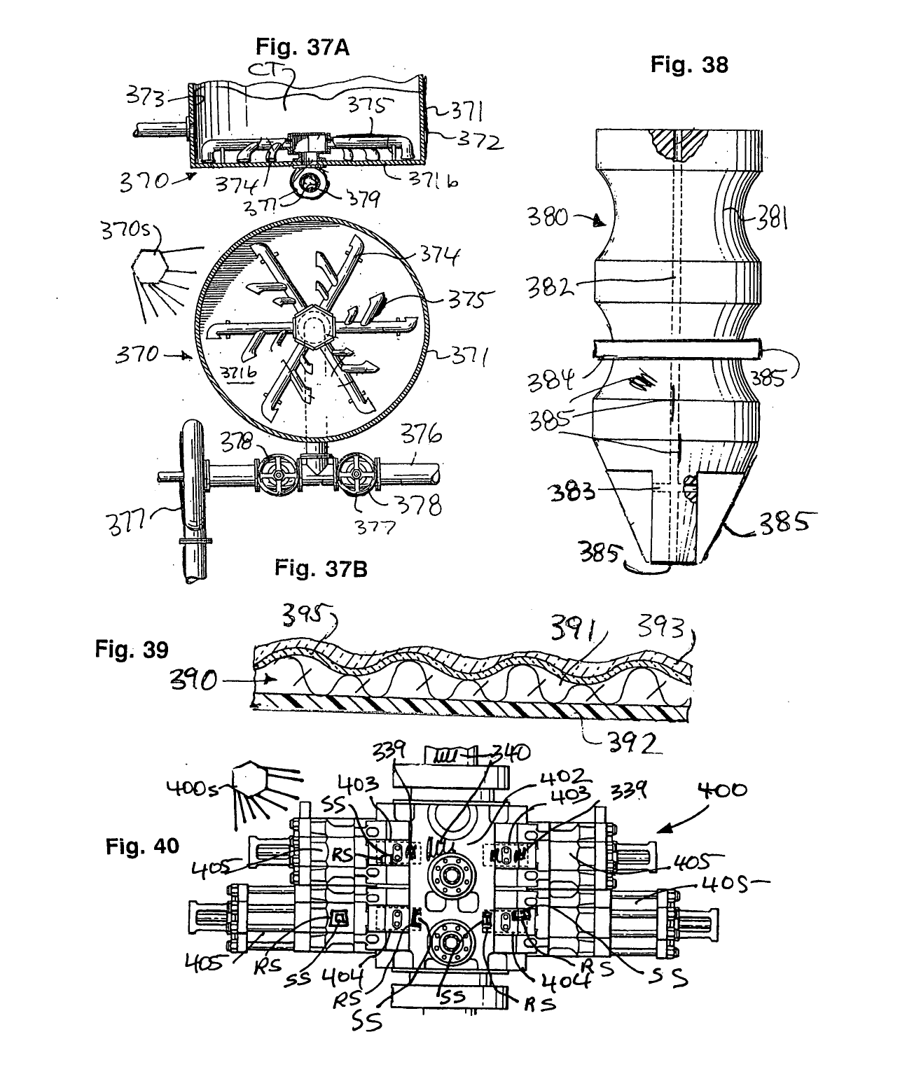

FIGS. 37A and 37B show a storage tank system 370 according to the present invention which has tank walls 371, bottom 371b, with coatings 372 and/or 373 according to the present invention. Optional movement apparatus 374 within the tank has coating 375 on its parts. Piping 376 has coating 377 according to the present invention which includes valves 378 and exit pipe 379—any and all of which may be coated according to the present invention. A system 370s according to the present invention provides the voltage to the coating(s) to heat the coating(s). The coatings are not shown to scale (as is true of all the drawings herein). The coating(s) may be used to control the viscosity of the contents CT of the tank. In certain aspects, the exterior coating and/or the interior coating, in contact with the earth, serve as a ground for the tank system, providing a flow path for electrons impressed on the tank to the ground. Any coating disclosed herein may be used for such grounding.

FIG. 38 shows a pipeline pig 380 according to the present invention which has a body 381, internal passageways 382, 383, and a removable disc (or discs) 384. Any or all of these parts may have a coating 385 according to the present invention partially or totally covering the part. A system 380s according to the present invention provides the voltage to the coating(s) to heat the coating(s). Suitable conductors may be provided within a pipeline, e.g., but not limited to the conductor CD shown in FIG. 36, for transmitting electricity to the pig 380, The coatings are not shown to scale (as is true of all the drawings herein).

FIG. 39 shows a fabric 390 according to the present invention which has a fabric layer (woven or unwoven) 391, an optional backing 392, and an optional wear layer 393. The fabric 390 is coated with a coating 395 according to the present invention partially or totally. A system 390s according to the present invention provides the voltage to the coating to heat the coating. The coatings are not shown to scale (as is true of all of the drawings herein).

It is within the scope of the present invention to provide a coating according to the present invention on a blowout preventer and to its internal and/or external parts and/or surfaces, including, But not limited to, its rams, actuators, seals, seal recesses, seal mount structures, and internal and external surfaces. The present invention is applicable to closing BOP's and to tubular-severing BOP's. As with other items, apparatuses, machines, and things described above according to the present invention, the provision of such a coating according to the present invention provides for, among other things, the selective heating and/or deicing of part or all of an item, etc.

FIG. 40 shows a blowout preventer 400 according to the present invention which has a body 402 with a plurality of spaced-apart rams 403 and 404 which are moved by actuators 405. According to the present invention, the body (inside and/or outside), parts, seal(s), and/or the rams are coated with a coating 339 or 340 according to the present invention (shown on some parts in exaggerated size). Any seal of any part of a blowout preventer according to the present invention may have a seal in a respective recess or mount with the seal and/or the recess coated with a coating (shown partially in exaggerated size) according to the present invention (see. e.g., the seals SS shown schematically in recesses RS to represent all seals and/or any seal of the preventer 400, seal of a ram, and/or seal of an actuator). A system 400s according to the present invention provides the voltage to the coating(s) of the blowout preventer 400 to heat the coating. It is within the scope of the present invention to delete the coating from any part of the blowout preventer 400 or to coat only one of the parts or surfaces.

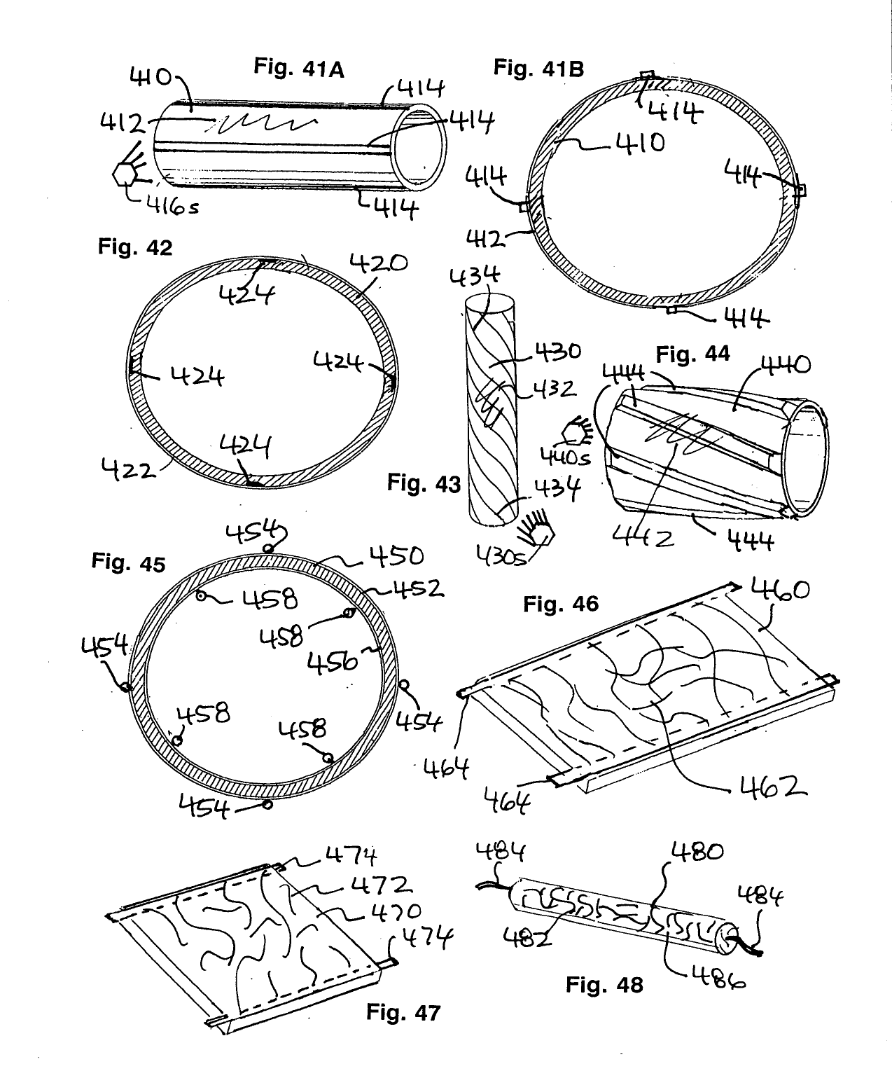

FIGS. 41A and 41B show a tubular 410 according to the present invention which has a coating 412 (not to scale) on its exterior (any coating according to the present invention) and leads 414 to which a voltage is applied by a system 416s. Any desired number of leads may be used, one or more. In one particular aspect, the tubular 410 is pipe which, in one aspect is rubber, PVC, or plastic “PEX” or crosslinked polyethylene and the leads are copper (e.g., copper wires or pieces of copper stock cut to size and glued to the surface of a pipe; e.g., but not limited to, a plastic pipe three-quarters of an inch in OD). The system 416s may be any system disclosed or referred to herein for applying a voltage. Any tubular according to the present invention may have its interior, exterior, or both coated with PTFE or with some other non-stick material.

In certain embodiments with copper wire leads as described above on PVC pipes and on rubber pipes, coated exteriorly with a coating having five percent multi-walled nanotubes by weight, electrical resistances of 0.632 k.OMEGA. and 1.25 k.OMEGA. were measured for PCV pipes (three quarters of an inch OD); and electrical resistances of 0.895 k.OMEGA. and 1.155 k.OMEGA. were measured for rubber pipes (three quarters of an inch OD). The temperature of these pipes increased with heating time as current was applied to the pipe's leads. For the PVC pipes the temperature over about fifteen minutes increased from about sixty-two degrees F. to about one hundred eighteen degrees F. (pipe with resistance of 0.632 k.OMEGA.—which is kΩ) and from about sixty-two degrees F. to about one hundred degrees F. (pipe with resistance of 1.25 kΩ). For the rubber pipes the temperature over about fifteen minutes increased from about sixty-two degrees F. to about seventy eighteen degrees F. (pipe with resistance of 0.895 kΩ. and from about sixty-two degrees F. to about seventy degrees F. (pipe with resistance of 1.15 kΩ).

As shown in FIGS. 42-45, it is within the scope of the present invention to use one, two, three, four or more leads or electrical contacts with an embodiment of the present invention (and with any embodiment disclosed herein) and, with tubulars (e.g., pipe, tubing, casing, risers, drill collars, drill pipe) the leads may be non-straight with respect to a longitudinal axis of the tubular. Also, it is within the scope of the present invention to coat the inside of a tubular, the outside of a tubular, or both. Contacts (any leads or contacts disclosed herein) may be on top of a coating or they may be coated by the coating.

FIG. 42 shows a tubular 420 according to the present invention with an exterior coating 422 which coats a plurality of leads 424 (not to scale) which are on the exterior of the tubular 420 and are in contact with the coating 422.

FIG. 43 shows a tubular 430 according to the present invention with an exterior coating 432 (not to scale) which coats a plurality of leads 434 which are in contact with the coating 432. The leads 434 wrap partially around the tubular 430 in a helical pattern. A system 430s applies voltage to the tubular's leads.

FIG. 44 shows a tubular 440 according to the present invention with an exterior coating 442 (not to scale) which coats a plurality of leads 444 which are in contact with the coating 442. The leads 444 are slanted with respect to the tubular 440 and wrap partially around the tubular 430. A system 440s applies voltage to the leads 444.

FIG. 45 shows a tubular 450 according to the present invention with an exterior coating 452 on top of which are a plurality of leads 454 which are in contact with the coating 452 and an interior coating 456 on top of which are a plurality of leads 458. As is true for any embodiment herein in which there are multiple leads, either or both of the leads 454, 458 may be under their respective coatings; or alternating leads for one coating may be coated or on top of a coating.

FIG. 46 shows an item 460 according to the present invention with a top coating 462 which also coats leads 464 which are in contact with the coating 462. In one aspect the item 460 was a glass slide, one inch by three inches, and the leads were aluminum with the coating containing four percent by weight multi-walled carbon nanotubes. This coating, in one aspect, due to the inclusion of the nanomaterial, was relatively viscous and was spread on the slide using a spatula, mechanical draw down film cutting device, or putty knife. This item had an electrical resistivity of about one hundred k.OMEGA. and was heated from seventy degrees F. to about one hundred and ten degrees F. with five minutes of applied current at six hundred volts.

FIG. 47 shows an item 470 according to the present invention with a top coating 472 which also coats leads 474 which are in contact with the coating 472. In one aspect, an item 470 was a six-inch by six-inch piece of HDPE, about one eighth of an inch think, spray coated with a coating four to six mils thick. The coating was sprayed to a thickness of about 0.04 inches and was a three part resin system with four percent multi-walled carbon nanotubes by weight. The coating three part resin system was polyisocyanate with a fluorinated polyol additive and cured with a dibutyl tin catalyst. Optionally any suitable conventional polyol, fluorinated or not, may be used. Such an embodiment heated sufficiently upon the application of an electrical current to its leads to provide de-icing. In a larger embodiment, with a piece of HDPE about twelve inches by twelve inches, the weight percent of nanotubes in the coating was five percent and similar heating was achieved.

FIG. 48 shows a protector 480 according to the present invention which has a body 486 and an exterior coating 482. Leads 484 extend from opposite ends of the body 486. In one aspect, the body 486 operates as an anode which efficiently enables impressed current cathodic protection due to the conductivity of the conductive nanomaterial coating. The body may, according to the present invention, of any desired shape, configuration and dimensions. In certain aspects, the coating 482 is applied to a thickness of about ten mils. In one particular aspect, the coating is as the coating described above with 5% carbon nanotubes by weight. For example, a body of metal, metal alloy(s), mixed metal oxide, copper, and/or graphite may be used (and/or of any known metal used for cathodic protection). By applying electrical current (e.g., referred to as an “impressed current” or “impressed electrons”) to the leads of the body, the underlying metal is protected from corrosion due to water exposure and/or chlorine gas generation (typical byproducts of impressed current cathodic protection) which can result in prior systems in the sacrificial corrosion of unprotected metal or graphite anodes. Any coating according to the present invention may be used for the coating 482. Any coating depicted in the drawings hereof with non-straight squiggly lines and/or with a dark mass are not to scale and are understood to cover an entire surface or part unless stated otherwise. Any embodiment not shown with a current application device or apparatus may have any such device or apparatus as disclosed or referred to herein; and any coating herein, without a current application device, may be used as a protective coating.

The present invention, therefore, in at least some embodiments, provides methods for heating including: applying a heatable coating onto at least part of an item; applying electric current to the heatable coating thereby producing heat in the heatable coating and heating at least part of the item; the heatable coating having base material, nanomaterial dispersed in the base material, the nanomaterial comprising electrically conductive nanomaterial so that when the electric current is applied, if flows through and heats the nanomaterial. Such methods may include, in any possible combination, one or some (any possible number) of the following: wherein the nanomaterial is one of or a combination of two of nanotubes, nanoribbons, nanographene, transformed nanomaterial, carbon nanomaterial, and carbon nanotubes; wherein the coating on all or on at least part of the item is between 0.0001 and 1 inch thick; wherein the nanomaterial, by weight, is at least 1% of the coating; wherein the nanomaterial, by weight, is at least between 0.1 and 32% of the coating; wherein the coating has an electrical resistivity due to the nanomaterial which is in a range which is one of between 10 to 100 Ω/cm or g*.OMEGA./cm and between 0.001 to 100 Ω/cm or g*.OMEGA./cm; wherein the base material is one of or a combination of two or more of glass, natural fabric, synthetic fabric, metal, elastomer, wood, plastic, composite, polymer, thermoplastic material, thermoset material, and high density polyethylene; wherein the coating is applied by one of dipping, spraying, spreading, trowelling, brushing, pouring, bonding, fusion bonding, and electrostatic coating; wherein the electrical current is one of direct current and alternating current; wherein the coating includes a resin material; wherein the item has a body with an outer surface and the at least part of the item is the outer surface of the body; wherein the coating further comprises two or at least two spaced-apart electrical contacts to which the electrical current is applied; wherein the nanomaterial is dispersed in the coating prior to application of the coating to the item by a method which is one of stirring, blending, mixing and sonication; wherein the item has ice thereon and the coating is heated sufficiently to melt the ice; wherein the coating (e.g., an exterior coating connected the item and to ground or an interior coating connected to the item and to ground) provides an electrical ground for the item; wherein the coating includes one of resin system, film, paint, and adhesive; wherein an electrical system provides the electrical current to the coating; wherein the electrical system includes a power supply and a voltage regulator; wherein the electrical system includes a sensor for sensing an event and a current source to apply electrical current in response to a sensed event, the event being one of ice formation and a predetermined state of temperature change; the base material is a cathodic protection material; and/or wherein the item is all or at least a portion of an airplane, an airplane part, wing, edge of a wing, propeller, part of a propeller, turbine blade, turbine blade edge, tower, wind power generator, pipeline, pipeline interior, pipeline exterior, bridge, part of a bridge, bridge deck, cable, support structure, ship, ship hull, boat, boat motor, vehicle, rail, automobile, truck, trailer, recreational vehicle, drilling system, pipe, tubular, casing, blowout preventer, seal, drilling equipment, drilling structure, drilling rig, conduit, offshore rig, centrifuge, shale shaker, screen, screen support, heat exchanger, finned tube, jet engine, tank, fabric, and cathodic protection structure.

The present invention, therefore, in at least some embodiments, provides heatable coatings for application to an item, such a heatable coating including: base material, nanomaterial dispersed in the base material, and the nanomaterial including electrically conductive nanomaterial. Such methods may include, in any possible combination, one or some (any possible number) of the following: wherein the nanomaterial is one of or a combination of two of nanotubes, nanoribbons, nanographene, transformed nanomaterial, carbon nanomaterial, and carbon nanotubes, wherein the coating is between 0.0001 and 1 inch thick, wherein the nanomaterial, by weight, comprises at least 1% of the coating, wherein the coating further comprises at least two or two spaced-apart electrical contacts to which the electrical current is applied, and/or wherein the coating includes one of resin system, film, paint, and adhesive, wherein the coating has an electrical resistivity due to the nanomaterial which is in a range which is one of a range between 10 to 100 Ω/cm and a range between 0.001 to 100 Ω/cm, wherein the base material is one of or a combination of two of glass, natural fabric, synthetic fabric, metal, elastomer, wood, plastic, composite, polymer, thermoplastic material, thermoset material, and high density polyethylene, and/or wherein the coating is applied by one of dipping, spraying, spreading, trowelling, brushing, pouring, bonding, fusion bonding, and electrostatic coating, and/or wherein the coating includes a resin material, film paint or adhesive.

The present invention, therefore, in at least some embodiments, provides an item coated at least partially with a coating, which is in one aspect a heatable coating, the coating including: base material, nanomaterial dispersed in and/or on the base material, and the nanomaterial in one aspect including electrically conductive nanomaterial, optionally the nanomaterial aligned or not. Such an item may be one of or a portion of an airplane, an airplane part, wing, edge of a wing, propeller, turbine blade, turbine blade edge, tower, wind power generator, pipeline, pipeline interior, pipeline exterior, bridge, bridge deck, cable, support structure, ship, ship hull, boat, boat motor, vehicle, rail, automobile, truck, part trailer, recreational vehicle, tubular, interior of a tubular in whole or in part, exterior of a tubular in whole or in part, drilling system, pipe, tubing, casing, blowout preventer, seal, drilling equipment, drilling structure, drilling rig, conduit, offshore rig, centrifuge, shale shaker, screen, screen support, heat exchanger, finned tube, jet engine, tank, fabric, and cathodic protection structure.

The present invention provides systems for heating a thing and, in some aspects for deicing applications which include a power supply, connections and a coating on the thing, the coating including a composite of embedded nanofibers in a polymer matrix. In certain aspects, such a composite is a composite as disclosed in U.S. Pat. No. 7,897,248. Such a composite may be made by incorporating nanofibers in a plastic matrix forming agglomerates, and uniformly distributing the nanofibers, e.g., but not limited to, by exposing the agglomerates to hydrodynamic stresses. The hydrodynamic stresses force the agglomerates to break apart. In combination or additionally elongational flow is used to achieve small diameters and alignment. The composite may be a nanofiber reinforced polymer composite system that includes a plurality of nanofibers that are embedded in polymer matrices in micron size fibers. Such nanofibers may be fibrils with diameters of 100 nm, multiwall nanotubes, single wall nanotubes and their various functionalized and derivatized forms. The method may include mixing a nanofiber in a polymer; and inducing an orientation of the nanofibers that enables the nanofibers to be used, e.g., to enhance mechanical, thermal and/or electrical properties. Orientation may be induced by high shear mixing and elongational flow, singly or in combination. Polymer may be removed from the nanofibers, leaving micron size fibers of aligned nanofibers.

A composite of the system described in the above paragraph may be a composite including: a polymer matrix reinforced with carbon nanotubes, wherein the carbon nanotubes are dispersed in the polymer matrix; and wherein the carbon nanotubes are integrated into the polymer matrix; wherein the integration includes alignment of the carbon nanotubes and formation of at least one type of linkage to at least a sidewall of the carbon nanotubes; wherein, optionally, the at least one type of linkage is one of a linkage between carbon nanotubes and the polymer matrix, between the carbon nanotubes, and/or combinations thereof.

In certain aspects, the present invention provides a method for heating including applying a heatable coating onto at least part of a thing or substantially all of it, applying electric current to the heatable coating thereby producing heat in the heatable coating and heating at least part of the thing or substantially all of it, the heatable coating including material which may be a composite as described above.

In certain aspects, the present invention provides new systems and coatings which use new and nonobvious forms of the subject matter of U.S. Pat. No. 7,938,991. These coatings may be used for any of the purposes described herein. In one particular aspect, the present invention provides a method for heating including applying a heatable coating onto at least part of a thing, the method including: a) introducing carbon nanotubes (“CNTs”) some of which at least are in bundles and prepolymer molecules into a solvent to form a solvent mixture, e.g. by dispersing and/or dissolving amounts of CNTs in order to form the solvent mixture, wherein the solvent mutually: i) dissolves prepolymer molecules; and/or ii) achieves molecular penetration into bundles of CNTs causing them to at least partially expand and/or disentangle; and b) atomizing the solvent mixture; and applying (e.g., but not limited to spraying) the solvent mixture onto a thing (e.g., a base, an item, a surface). In other aspects, solvent is removed from the mixture before application to the thing; and, optionally, the method includes at least partially curing (or fully curing) the prepolymer (at least somewhat preventing rebundling and/or reentanglement of the CNTs) to provide solid polymer/CNT particles; and, optionally, the applying then includes depositing resulting solid polymer/CNT particles on a the thing, in one aspect causing the solid polymer/CNT particles to agglomerate while completing their cure, to form a polymer/CNT coating on the thing of desired characteristics and dimensions (e.g., but not limited to, of any thickness disclosed herein). Optionally, the CNTs are single-walled and/or functionalized with a desired functionalization (e.g., but not limited to, any functionalization mentioned or referred to herein or in any reference herein). Optionally, the prepolymer is one of or a combination of epoxy resins, unsaturated polyester resins, and vinyl ester resins. Optionally, the step of introducing involves a technique which is one of sonication, mechanical shear, heating, and combinations thereof and/or the step of rapidly removing solvent comprises exposure to a condition selected from the group consisting of heating, mechanical pumping, evacuation, and combinations thereof. Optionally, the solid polymer/CNT particles have a size that ranges from about 1 micron to about 100 microns and/or are aligned. Such a method may also include applying electric current to the coating (any described in this paragraph) thereby producing heat in the coating and heating at least part of the thing on which the coating is.

In certain embodiments, a thing is made from a composite produced according to U.S. Patent 7,938,91 and a coating according to the present invention is applied to the thing (any coating according to the present invention) and, in certain aspects, the coating has an electrical current passed therethrough, e.g. as in any system herein for imposing a potential difference across a thing.

Coatings according to the present invention, in certain aspects, may be used as thermoelectric materials. It is within the scope of the present invention to apply heat to any suitable coating according to the present invention to thermoelectrically produce electric current; and to apply heat to any suitable item or suitable thing coated, in whole or in part, with a coating according to the present invention to thermoelectrically produce electric current. Thermoelectric materials act as energy conversion materials in which electric energy is generated when a temperature difference is applied between two parts or two ends of the material. It is within the scope of the present invention for the heat to be applied using any suitable known heat source, heating system, or heating apparatus; and, e.g., the heat may be applied by applying microwaves to thermoelectric material and/or nanomaterial, or by inductively heating thermoelectric material and/or nanomaterial.

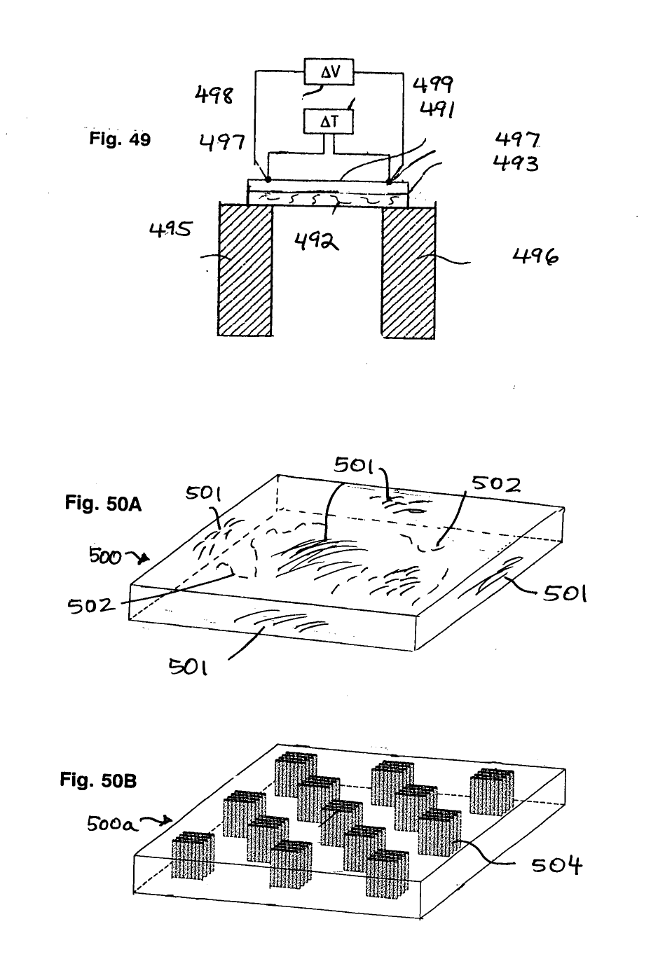

FIG. 49 shows a thing 353 with a coating 351 (neither to scale) with electrodes 357 in contact with the coating 351 (any suitable coating according to the present invention). A heater 355 and a cooler 356 apply a temperature difference between the two electrodes. This temperature difference causes current to flow between the electrodes and an electric potential can be measured between the electrodes with the voltmeter 358. A temperature sensor system 359 can measure the temperature difference between the electrodes. In one particular aspect, the thing 353 is a glass substrate and the electrodes are patterned gold electrodes as in U.S. Patent Application Pub No. 2013/0276851. Optionally, the thing 353 has nanomaterial 352 therein which may be any suitable nanomaterial disclosed herein. Optionally, the coating 351 may enclose the thing 353 completely, may be only on one surface thereof, or may be on multiple surfaces thereof.

FIG. 50A shows a thing 500 with coating 501 (any suitable coating according to the present invention). In certain aspects, the thing 500 is one embodiment of the thing 353 of FIG. 49. The thing 500 may be used as a thermoelectric material for producing electric current. Optionally, the thing 500 has nanomaterial 502 dispersed therein. The coating 501 is shown as covering substantially all of the surface of the thing 500, but it may cover less than all the surface.

FIG. 50B shows an embodiment 500a of the thing 500 which has discrete amounts of nanomaterial 504 spaced-apart within the thing 500. In one particular aspect, the thing 500a is a made of thermoelectric composite material as in U.S. Patent Application Pub. No. 2013/0153819 and the nanomaterial 504 is one of or a combination of the nano-carbon material units disclosed in this patent application. In certain particular embodiments, the thermoelectric composite material includes: a thermoelectric matrix which is a thermoelectric material; and a plurality of nano-carbon material units located in the thermoelectric matrix and spaced apart from each other, with any suitable spacing, including, but not limited to about 50 nm to 2 micrometers, or between 200 nm to 1 micrometer, or between 400 nm to 700 nm. In such a thermoelectric composite: the thermoelectric material may be one of or a combination of bismuth telluride, antimony-doped bismuth telluride, selenium-doped bismuth telluride, zinc antimonide, or half-Heusler alloys; the nano-carbon material units may be one of or a combination of carbon nanotubes, graphite, or graphene; they may be in columnar shapes and/or embedded in the thermoelectric matrix along a single direction or along different directions respectively, and/or they may be arranged in a one-dimensional array, a two-dimensional array, or a random manner in the thermoelectric matrix.

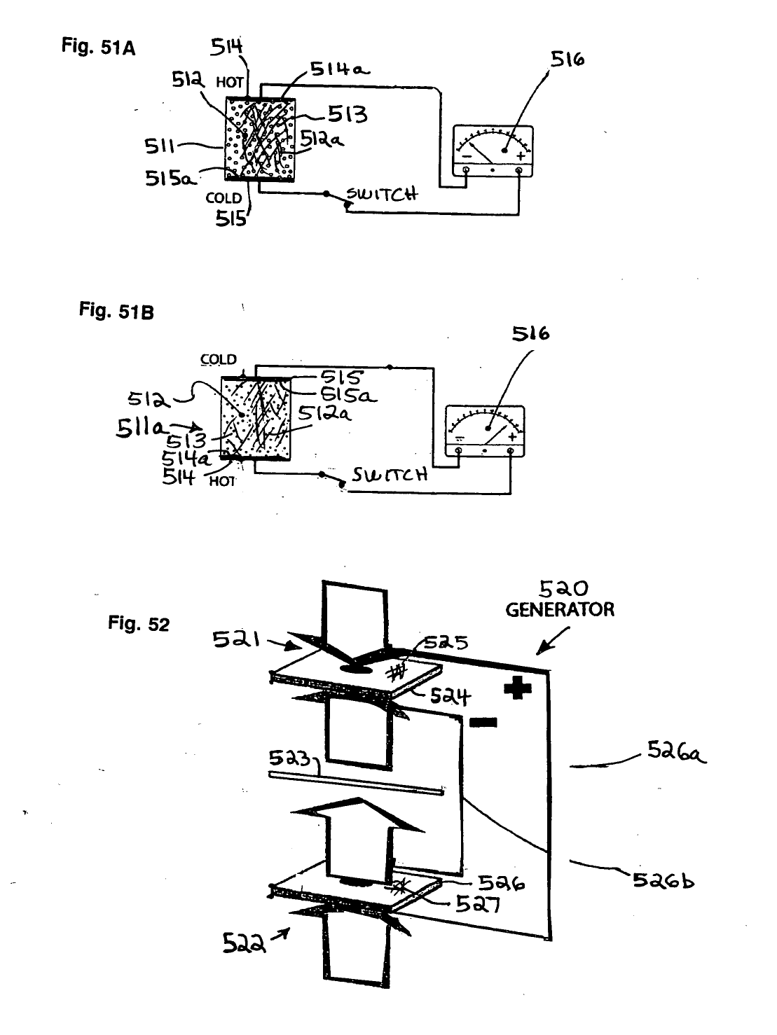

FIGS. 51A and 51B show thermoelectricity generators in which heat, applied to one side of an assembly and with the second side of the assembly maintained at constant temperature or cooled, is converted into electrical energy. The cooling of a part of the assembly shown in these figures may be deleted or the heating of a part of the assembly may be deleted.

As shown in FIG. 51A a cell 511 of thermoelectric material 512 with or without nanomaterial 513 dispersed therein has a hot side (or electrode) 514 and, optionally, a cooled side (or electrode) 515. According to the present invention, the hot side is coated with a coating 514a according to the present invention (any suitable coating disclosed herein, e.g. for enhancing thermal conductivity and/or electrical conductivity); the cold side is coated with a coating 515a according to the present invention (any suitable coating disclosed herein, e.g. for enhancing thermal conductivity and/or electrical conductivity); and/or the material 512, in whole or in part, is coated with a coating 512a according to the present invention (any suitable coating disclosed herein, e.g. for enhancing thermal conductivity and/or electrical conductivity). For certain embodiments this type of system is known generally as a “Seebeck” thermoelectric device. A power/voltage indicator 516 indicates the amount of power or electrical potential generated by the cell 511. The hot electrode is generating a negative potential and the cold electrode is charged positively.

As shown in FIG. 51B a cell 511a is like the cell of FIG. 51A (like numerals indicate like parts) but with the HOT and COLD sides reversed. The hot electrode is generating a positive potential and the cold electrode is charged negatively.

Thermoelectric generators in general, and any disclosed herein, may be operated to produce heating of a material by passing current through electrically conductive material of the thermogenerator. A generator operated in such a manner is called generally a “Peltier cell” or a “Peltier device.”

FIG. 52 shows an generator apparatus 520 which has a Seebeck device 521 connected with wires 526a and 526b to a complementary Peltier device 522. A thermal gradient applied across the Seebeck device 521 generates electromotive force which is applied to the Peltier device below which can act as a cooling device.

Optionally an adiabatic wall 523 is used between the Seebeck and Peltier devices which can provide for both devices to coexist in thermal independent equilibrium states for any temperatures involved and, optionally, with the common adiabatic wall maintained at constant temperature, e.g. by induced heat removal with heat sinks, flowing gas, fluid or by alternative means, such as solid state, plasma or active refrigerants. The adiabatic wall may also provide good electrical connection between the electricity-producing Seebeck device and the cooling element, the Peltier device.

The Seebeck device 521 has thermoelectric material 524 (with or without nanomaterial therein) and is coated, in whole or in part, with a coating 525 according to the present invention (any suitable coating disclosed herein, e.g. for enhancing thermal conductivity or for enhancing electrical conductivity, or both). The Peltier device 522 has thermoelectric material 526 (with or without nanomaterial therein) and is coated, in whole or in part, with a coating 527 according to the present invention (any suitable coating disclosed herein, e.g. for enhancing thermal conductivity or for enhancing electrical conductivity, or both).

Optionally, the cells, generators, and devices of FIGS. 51A, 51B and 52 are as disclosed in U.S. Patent Applications Pub. Nos. 2005/0236028 and/or 2005/0139248, but with the improvements noted herein according to the present invention.

It is within the scope of the present invention to provide a wall, a window, a pane for window, and a blind or shade for a window that has a coating according to the present invention for heating or cooling. Any such wall, etc. has a power supply or supplies for each coating associated with the wall, etc.

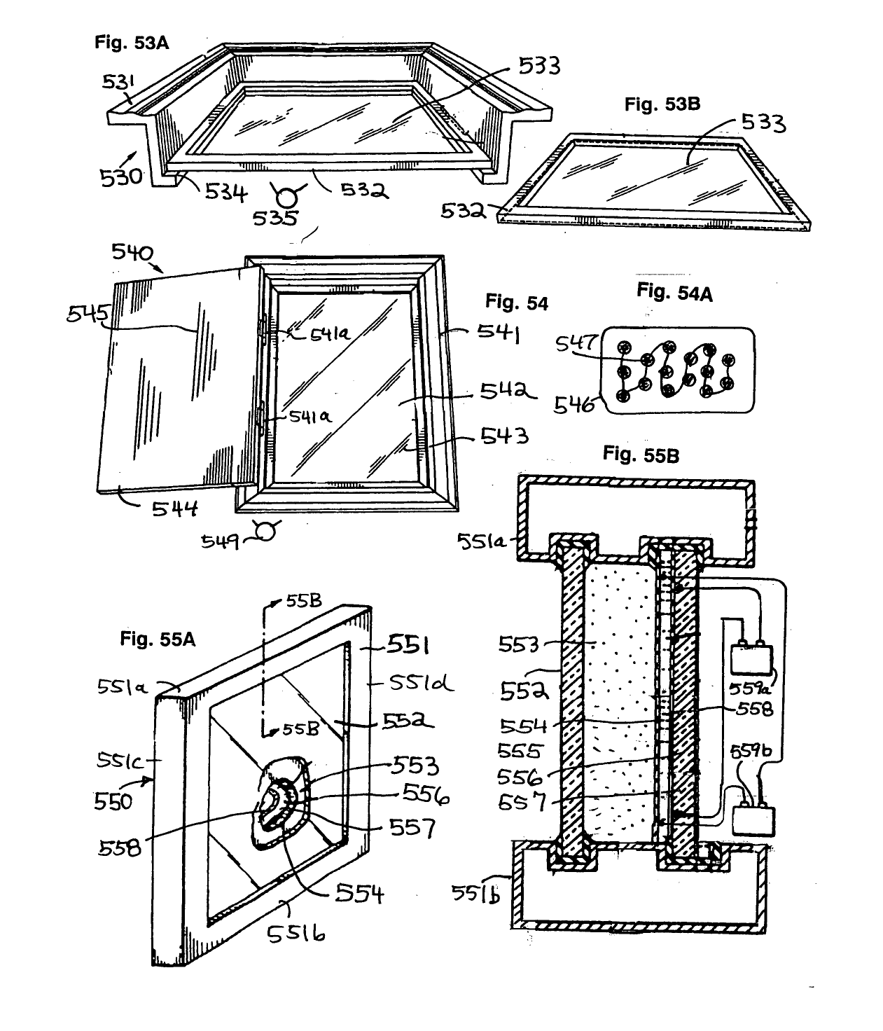

FIG. 53A shows a window unit 530 according to the present invention with a frame 531 having a lip 534 on which is positioned a pane 532 according to the present invention. The pane 532 has one surface with a coating 533 (any suitable coating according to the present invention). A power supply 535 is connected to the pane 533 for producing electric current in the coating to heat the coating. The pane 533 (FIG. 53B) may be removably or releasably positioned on the lip 534.

FIG. 54 shows a window unit 540 according to the present invention which has a frame 541 to which is connected a pane 542 which, optionally, has a coating 543 according to the present invention. A panel 544 according to the present invention is movably connected to the frame 541, e.g., with hinges 541a, and can be moved into and out of position parallel to the pane 542. A power supply 549 provides power for heating the panel 544 by passing an electric current through a coating 545 on the panel 544 (any suitable coating according to the present invention).

It is within the scope of the present invention for any coating according to the present invention to be applied to a pane or panel in such an amount that the pane or panel is at least somewhat translucent or at least somewhat transparent and/or with such a nanotube loading to achieve some transparency or translucency. It is within the scope of the present invention to apply a coating to a pane or panel so that some areas or parts of the pane or panel have no coating so that the pane or panel is partially transparent or partially translucent, yet with sufficient coating areas connected appropriately so that desired heating of the pane or panel can be achieved. FIG. 54A shows a pane 546 with coated areas 547 which are electrically connected for heating, but which do not cover sufficient space on the pane 546 so that some of the pane's area is transparent or translucent.

FIG. 55A shows a window unit 550 according to the present invention that has a frame 551 with mounted multiple panes 552 and 556 and a gap 553 between the panes, e.g. filled with air or with an inert gas. The frame 551 has a top 551a, a side 551c, a side 551d, and a bottom 551b. Typical frame pane mounting structure and seals may be used. The pane 556 has a coating 557 (any suitable coating according to the present invention) which is connected to a power supply 559b (FIG. 55B) for providing electric current to the coating 557. A sheet 554, spaced apart from the pane 556, is connected to the frame 551 and is positioned between the panes 552 and 556. A power supply 559a provides electric current to the coating 554.

It is within the scope of the present invention to provide a sheet or panel, which may or may not be flexible, as part of a window unit, the sheet or panel to be placed adjacent or near a pane or to be placed in a gap between two panes. The sheet or panel has associated with it a power supply for providing electric current for heating nanomaterial in the or on the sheet or panel. As is true for any wall or window unit according to the present invention, the power supply may be any suitable known power supply, battery, or solar power system. Also any suitable coating on any pane or panel according to the present invention may be heated using microwaves or heated via inductive heating.

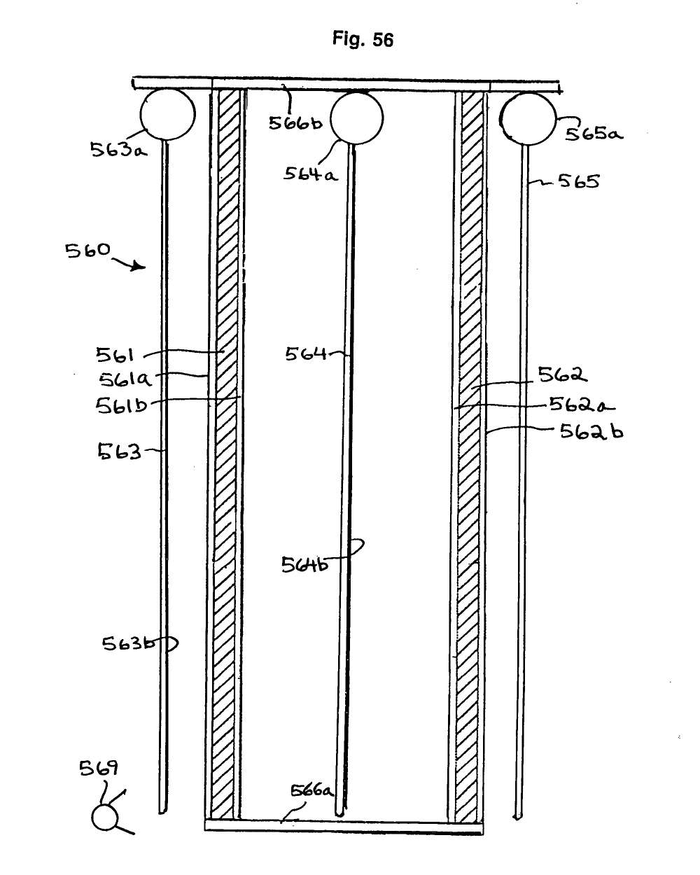

FIG. 56 shows in crosssection a window unit 560 (which may, e.g., be configured generally like those of FIGS. 53A-55B with a frame, e.g. such as those of these embodiments) which has a frame top 566b and a frame bottom 566a between which are two spaced-apart panes 561 and 562. On one side of the pane 561 is a sheet 563 according to the present invention which has a coating 563b (any suitable coating according to the present invention) which has associated with it a power supply 569. In one aspect, the sheet 563 is flexible and is supplied from and returnable into an apparatus 563a connected to the frame top 566b.

Between the panes 561 and 562 is a sheet 564 according to the present invention which has a coating 564b (any suitable coating according to the present invention) which may be associated with the power supply 569 or with its own dedicated power supply. In one aspect, the sheet 564 is flexible and is supplied from and returnable into an apparatus 564a connected to the frame top 566b.

Spaced apart from the pane 562 is a sheet 565 according to the present invention which has a coating 565b (any suitable coating according to the present invention) which may be associated with the power supply 569 or with its own dedicated power supply. In one aspect, the sheet 565 is flexible and is supplied from and returnable into an apparatus 565a connected to the frame top 566b.

Any one or two of the sheets 563, 564, and 565 may be deleted.

Optionally, the pane 561 has a side coated with a coating 561a and/or a side coated with a coating 561b; and/or the pane 562 has a side coated with a coating 562a and/or a side coated with a coating 562b-each or all of said coatings being any suitable coating according to the present invention, and the coating(s) associated with the power supply 569 or each with its own dedicated power supply. “Power supply” includes any appropriate controls, leads, wires, switches, components, and device(s) for applying selected current and/or voltage to a coating.

It is within the scope of the present invention for any pane described above to be a wall or part of a wall; and for any wall to have any of the sheets described above used therewith or adjacent thereto.

It is within the scope of the present invention for any coating disclosed herein for any purpose or for achieving any goal to be insulated from other items or things with which the coating comes in contact or in proximity to which the coating is used. This insulation can cover the coating in whole or in part. It can be on only one surface of coating, part of one surface, on multiple surfaces, or on all surface of the coating. In certain aspects, this insulation is thermal insulation. In certain aspects, this insulation is electrical insulation. With respect to any metal item, thing or tubular, a coating according to the present invention may be electrically insulated with respect to the item, thing, or tubular. In certain aspects, for any coating of any surface of a tubular, of a tubular's thread, or of part of a thread, the coating may be thermally and/or electrically insulated from the tubular, surface, thread or thread part.

It is within the scope of the present invention to provide heat using a coating according to the present invention to facilitate the hardening, curing and setting (all collectively referred to herein as “curing” and hardened, set, or cured material collectively referred to as “cured”) of various materials such as, e.g., certain epoxies, resins (e.g., curable resin(s), such as a thermosetting polyester, epoxy or vinylester resin), composites, polymers, and cements, and things containing or impregnated with such materials such as, but not limited to, fabrics, fibrous masses, felts, sleeves, mats, and webs, woven or nonwoven.

Material to be cured is coated with a heatable coating according to the present invention, coated in whole or in part; material to be cured is in a container, conduit or structure which is coated, in whole or in part, with a coating according to the present invention; or material used to effect curing, such as steam or water, is heated, directly or indirectly, using a coating according to the present invention heated according to the present invention. The coating is heated in any way with any apparatus or system as disclosed herein. With appropriate controls, wirelessly or with wiring, this heating, in certain aspects, is done selectively, periodically, or as desired.

In certain particular aspects, the cure of cure-in-place pipe and cure-in-place liners and repair structures (some of which are called “CIPP”) are facilitated by heating the pipe, liner or structure, directly or indirectly, using a coating according to the present invention heated according to the present invention.

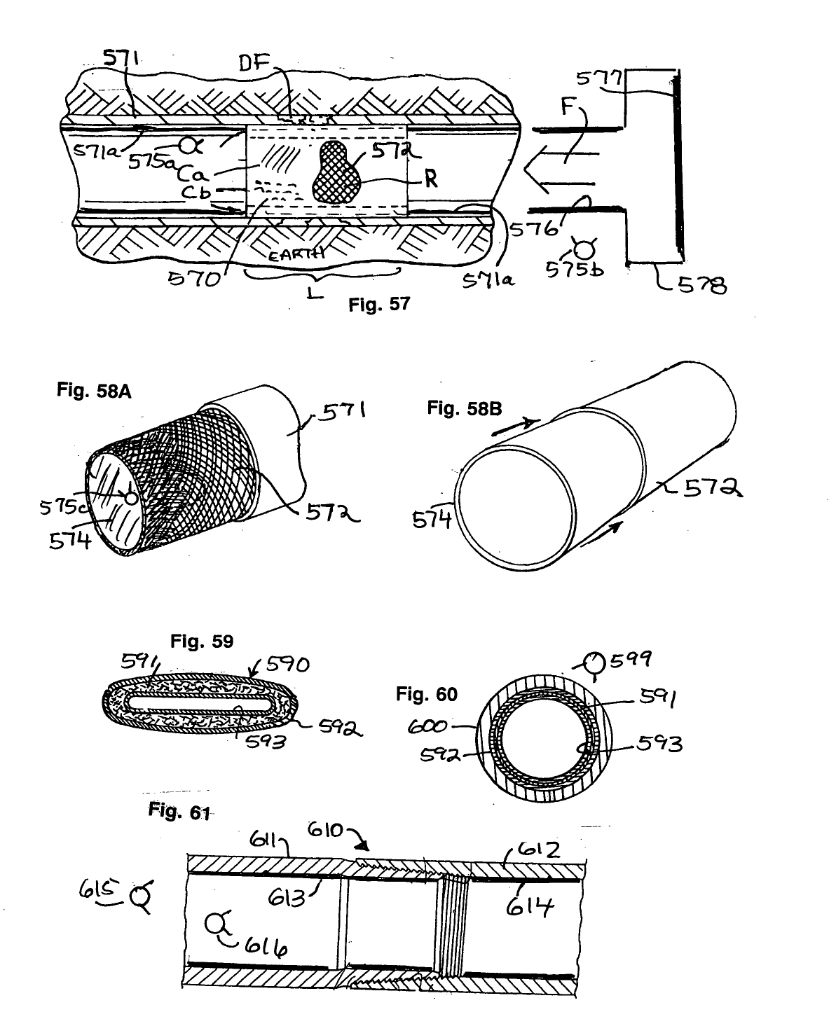

FIG. 57 illustrates the installation of a repair structure 570 in a pipe 571 shown as buried in the earth (but this pipe and any pipe or pipeline shown herein within the earth may be above ground). The repair structure 570 is located adjacent a defect DF has a layer of material 572 impregnated with resin R which is not yet cured when the repair structure 570 is inserted in the pipe 571 at a location L that is to be reinforced or repaired. Fluid F is pumped into the pipe 571 to the repair structure 570 to facilitate expansion of the repair structure at the location L and, in certain aspects, to facilitate in heating the resin R to cure it. The interior, the exterior, or both of the repair structure 570 may have a coating Ca and/or Cb according to the present invention which is used to heat the repair structure 570 to facilitate curing of the resin R.

In one aspect, a coating according to the present invention is in the form of a cylinder or sheet 574 (see FIGS. 58A, 58B) which is installed within the material 572 of the repair structure 570, either before the repair structure is positioned within a pipe or after the repair structure in positioned within a pipe. In certain aspects, the cylinder or sheet is flexible. Optionally, such a flexible coating is emplaced around the repair structure on its exterior; or such flexible coatings are on both the interior and the exterior of the repair structure.

In certain aspects, a power supply 575a in the pipe 571 provides power for a coating or coatings according to the present invention which are on or near the repair structure 570 and/or are on the pipe 571. Optionally a power supply 575b supplies power for a coating 576 (any suitable coating according to the present invention) on a conduit through which fluid F flows and/or for a coating 577 (any suitable coating according to the present invention) on equipment 578 which pumps, processes, transmits, and/or treats the fluid F. Optionally a power supply 575c is within and/or connected to the repair structure 570. Any suitable known power supply may be used as these power supplies 575a, 575b, and 575c (or as a power supply for any system or apparatus or coating herein) with any suitable known control and wiring (or wireless system), with or without a solar power supply.

Optionally, the pipe 571 has a coating 571a according to the present invention for, inter alia, inhibiting deposits on the pipe's interior, providing heat to cure a liner of the pipe, or to provide heat to facilitate curing of material—such as resin—in a repair structure.

FIG. 58A shows a pipe 580 with a liner 582 having a coating 584 according to the present invention. A power supply 589 provides electric power for the coating 584. As shown schematically in FIG. 58B, the coating 584 may be in the form of a flexible sheet manipulated into a shape suitable for insertion into the liner 582 and positioned therein as in FIG. 58A. Optionally the liner 582 is a repair structure according to the present invention or is any suitable known liner to which a coating according to the present invention has been added.

It is within the scope of the present invention to provide a repair structure 590, as shown in FIG. 59, which has resin-impregnable material 591 coated with an exterior coating 592 and, optionally, with a coating 593 on an interior surface of the material 591. Optionally, only the coating 593 is used. A power supply 599 provides power for the coating(s). FIG. 60 shows a tubular 600 with the repair structure 590 installed therein following curing of resin in the repair structure.

It is within the scope of the present invention to use a heatable coating according to the present invention to reduce, inhibit, or prevent hydrate formation and the formation of deposits on equipment and tubulars such as, but not limited to, the formation of deposits on tubulars and conduits used in drilling oil and gas wells and in the production of hydrocarbons from a well; e.g., but not limited to deposits (on an interior surface, exterior surface, or both) of paraffin, hydrates, asphaltenes, scale and other undesirable materials on tubing (including but not limited to production tubing and coiled tubing), drill pipe, couplings, connections, and casing in a well. Such coating(s) may also be used to facilitate connection and disconnection of tubulars, e.g., but not limited to both threaded and non-threaded tubulars. Such coatings, heated or not, can also reduce undesirable deposits.

FIG. 61 shows a production tubular 610, e.g., production tubing or production casing, according to the present invention which includes multiple connected pieces of tubulars, e.g. as the two threadedly-connected tubulars 611 and 612 (shown partially). Tubular 611 has an interior coating 613 and tubular 612 has an interior coating 614. Schematically indicated power supplies 615 and 616 are meant to illustrate the use of any suitable power supply as disclosed herein for use with the coatings 613 and 614. The coatings may be used to achieve any of the goals or purposes disclosed herein for such coatings. In certain particular aspects, it is to be noted that a coating adjacent all or part of a threaded area of a tubular may be used to facilitate threading or unthreading of mating tubulars by the application of heat to the threaded areas.

It is within the scope of the present invention to provide a coupling or connector for connecting two tubulars together which has a coating or coatings according to the present invention for heating the coupling or connector for facilitating connection of two tubulars, disconnection of two tubulars, make-up of a joint between two tubulars or break-out of a joint. Optionally, a threaded area of a threaded tubular may be coated with any coating disclosed herein; including, but not limited to, any coating herein for strengthening an area, protecting an area, of used for heating an area. Entire threads may be coated or only a portion of the threads may be coated; e.g., either less than all a threaded area longitudinal of a tubular may be coated, or for each hill and valley of a thread only part of the valley or only part of the top point of a hill of a thread may be coated. Similarly, any coating disclosed herein for use in heating an area may be used to protect an area or to strengthen an area.

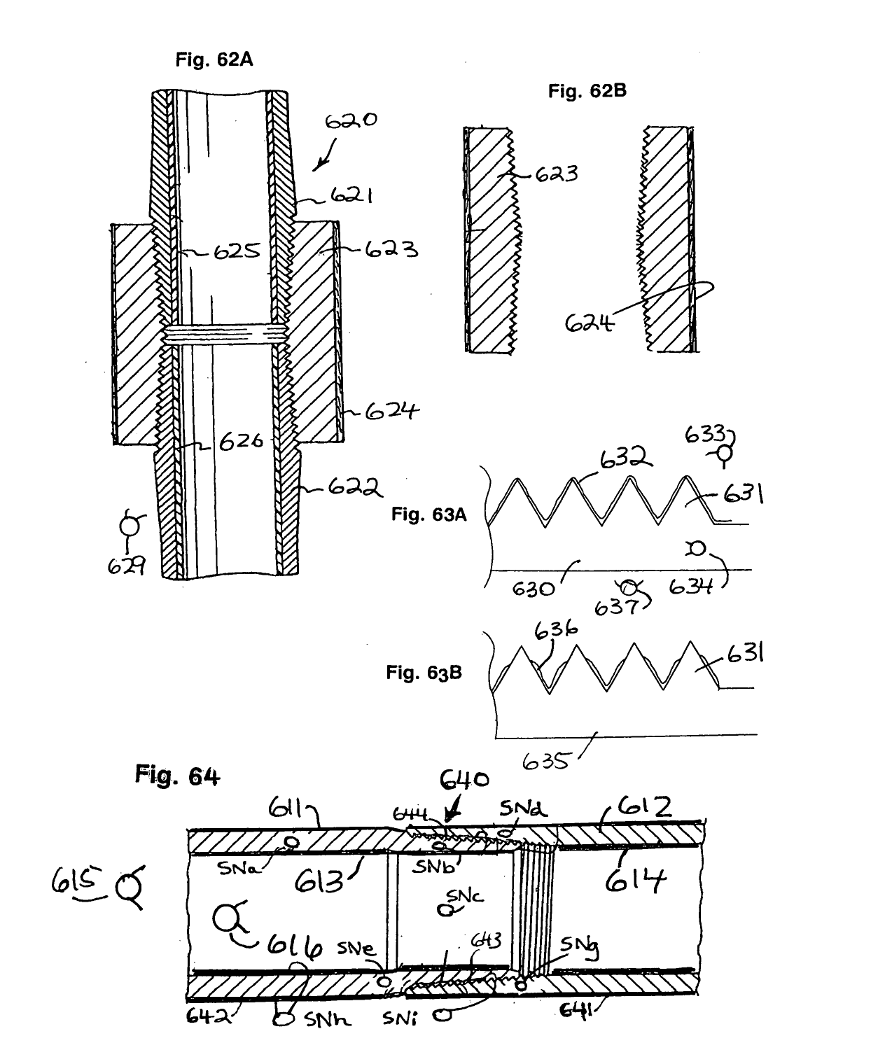

FIG. 62A shows a joint 620 according to the present invention which has two threaded tubulars 621 and 622 (shown partially) coupled together with a coupling 623. The coupling 623 (see FIG. 62B) has an exterior coating 624—e.g, as any coating according to the present invention as disclosed herein used for any purpose or goal as disclosed herein. A power supply 629, indicated schematically, represents any power supply disclosed herein, within or outside of the joint 620. Optionally a coating is on the interior of the coupling 623 encompassing the threaded area, extending beyond it, or adjacent the threaded area. Optionally, the tubular 621 has an interior coating 625 and/or the tubular 622 has an interior coating 626 (any coatings disclosed herein).

FIG. 63A shows a tubular 630 with a thread 631 with a coating 632 (any suitable coating according to the present invention) covering the threaded area, i.e., entire hill/valley structure of the threaded area is covered. Any suitable power supply may be used with the coating, e.g. a power supply 633 outside the tubular 630 and/or a power supply 634 within the tubular and/or a power supply 637 in a fluid passageway through the tubular.

FIG. 63B shows a tubular 635 with a thread 631 with a coating 636 (any suitable coating according to the present invention) covering part of the threaded area, i.e., part of the valleys of a hill/valley structure of the threaded area is covered. Any suitable power supply may be used with the coating.

It is within the scope of the present invention to provide a coating (any suitable coating according to the present invention) that is used: to inhibit the attachment of living things to an item or substrate; to inhibit the agglomeration of living things on an item or substrate; to inhibit the growth of living things on an item or substrate; to kill living things; to produce chemicals that kill living things; and/or to change the properties or characteristics of water around an object so that living things are killed therein or their growth, attachment, and/or agglomeration are inhibited or prevented, e.g., but not limited to, changing the acidity of water.

Such goals may be achieved by heating a thing and/or water in which the thing is submerged or water adjacent thereto, and/or by changing the chemistry of such water by applying a potential difference to a coating according to the present invention or by passing electricity through such a coating. These actions can, in some aspects, improve the operation or enhance the use of an electrically conductive submerged thing (surface, object, or item) that is continually or periodically caused to conduct an electric current in order to change the chemical or ionic character of water which is in contact with the submerged surface, etc. In saltwater, one such system produces gaseous chlorine at the submerged surface, etc. In freshwater, the acidity of the water adjacent the submerged thing is increased. In some such cases, biofouling deposits or growths are discouraged, inhibited, or prevented.

To achieve any of the goals stated in the previous paragraph, a coating according to the present invention may have nanomaterial therein that is nanotubes with a metal deposited thereon that inhibits or kills living things, a metal such as silver or copper. This metal may be on the interior, exterior, or any surface of a nanotube; and it can be positioned amidst a plurality of nanotubes that encompass it without it being bonded to or connected to any of the plurality of nanotubes. It may also be positioned within a nanotube without any such bonding or connection to the nanotube. In certain aspects, such a metal is controllably released from the nanotubes using any suitable known method, composition, material and/or structure so that the metal is released over a desired period of time and in a desired amount. In certain aspects, a dissolvable coating covers the metal and the coating dissolves according to a desired program over time so that desired controlled release of the metal is achieved.

In certain aspects, an electric current is periodically pulsed through such a coating to inhibit living things or to prevent them from attaching to, building up on, or agglomerating on an item or substrate. In certain aspects, a constant current is provided through the coating. In certain aspects, a constant low level electric charge is applied to the coating, e.g., but not limited to, at a voltage of less than 100 volts and a current of about 1 milliamp.

Any suitable nanotubes, in any suitable amount, in any configuration or shape in any coating described herein may be used as an indicator or sensor element in a sensor or sensor system in which a change in the nanotubes is monitored and then a signal or impulse indicative of the change is analyzed to provide information about the cause, implications of, and/or effect of the change. Any such coating may be on any suitable item, base, thing or substrate to serve as the indicator or sensor element.

For example, and not by way of limitation, any sensible and/or detectable change in the following of an amount of nanotubes in a coating according to the present invention may be the basis for use of them in a sensor: conductivity, temperature, shape, size, location, flexing, configuration, dimensions, and resistivity. Such nanotubes may be used to sense, measure, and/or indicate a change in, e.g., pressure, temperature, strain, gas presence, gas level, cracking, gas flow, pH, electromagnetic waves, force, liquid presence or not, fluid presence or not, liquid flow, fluid flow, presence of explosives, light, change in light, presence of light, intensity of light, crack presence, crack growth, electrical conductivity, magnetic field strength, electrochemical response or effect, or electrical resistivity.

Any of these parameters or effects may be sensed with respect to any item, substrate, or thing, or part thereof, disclosed in this application, including, but not limited to, those things and items shown in the drawing figures.

A typical sensor system according to the present invention includes a suitable amount of a coating according to the present invention; an apparatus, instrument or device for monitoring the coating; and analysis apparatus for receiving information from the instrument, etc. and for analyzing the information. In certain particular embodiments, the power supply or power system disclosed for any embodiment herein (which imposes a potential difference on a coating) is either replaced by or augmented by the instrument, etc. and/or the analysis apparatus.

In certain aspects, a suitable coating according to the present invention in a suitable amount, shape, location and configuration is used as a sensor, a nanomaterial element of a sensor, a nanomaterial part of sensor, or a nanomaterial component of a sensor system in a system as disclosed in these U.S. patent applications, Publication Nos.: 2014/0023116; 2014/0021067; 2013/0306491; 2013/0256627; 2013/0235375; 2013/0218050; 2013/0213140; 2013/0209991; 2013/0197319; 2013/0195140; 2013/0186177; 2013/0130230; 2013/0104665; 2013/0043140; 2013/0017611; 2012/0266685; 2012/0125084; 2012/0037306; 2011/0297541; 2011/0290672; 2011/0286889; 2011/0226066; 2011/0023608; 2010/0245808; 2009/0275143; 2009/0140167; 2008/0292531; 2008/0229839; 2008/0216558; 2008/0143351; 2008/0067619; 2007/0292601; 2007/0186665; 2007/0116628; 2006/0283262; 2006/0228723; 2005/0184641; 2005/0134296; 2005/0124020; 2005/0045477; 2005/0018274; and 2003/0087130—all said applications incorporated fully herein for all purposes.

It is within the scope of the present invention to have a sensor or a sensor element within or outside of an item or thing with a coating according to the present invention, or within a solid part of a thing or item that has a coating. In certain particular embodiments, the thing or item can be a cylinder, including, but not limited to, a tubular, including, but not limited to, casing, tubing, pipe, risers, conduit, and hoses.

FIG. 64 shows a tubular 640 according to the present invention with some parts and components like those of the tubular 610 of FIG. 61 (and like numerals indicate like parts). The tubular 640 and the tubulars 611 and 612 may be any known tubular—e.g., conduit, coiled tubing, pipe, casing, riser, or tubing. The tubular 640 has an exterior coating—coating 641 on tubular 612 and coating 642 on tubular 611, the threads of the tubular 611 have a coating 643, and the threads of the tubular 612 have a costing 644, all said coatings are coatings according to the present invention. Optionally, when the tubular is coiled tubing, there are no multiple tubulars like the tubulars 611 and 612, and no threaded ends of multiple tubulars; but it is within the scope of the present invention for the exterior, interior, or both of coiled tubing to have a coating according to the present invention and, optionally, any sensor system disclosed herein.

Any suitable sensor or sensor system may be used to sense a change in any of the coatings. Any sensor or sensor system, as desired, may be located in, outside of, or within the tubular 640. For example, a sensor system SNa is within the tubular 611; A sensor system SNb is within the tubular 611 in a threaded end thereof; A sensor system SNc is within the tubulars 611, 612 and can be connected to or attached to the tubular(s) or not. A sensor system SNd is within the threaded end of the tubular 612; a sensor system SNe is within the tubular 611 near a shoulder thereof; a sensor system SNg is within the threaded end of the tubular 612; a sensor system SNh is outside the tubular 640; a sensor system SNi is outside the tubular 612 (and 640). The sensor systems may monitor the coatings indicated by the lead lines from a sensor system to a coating.

In conclusion, therefore, it is seen that the present invention and the embodiments disclosed herein and those covered by the appended claims are well adapted to carry out the objectives and obtain the ends set forth. Certain changes can be made in the subject matter without departing from the spirit and the scope of this invention. It is realized that changes are possible within the scope of this invention and it is further intended that each element or step recited in any of the following claims is to be understood as referring to the step literally and/or to all equivalent elements or steps. The following claims are intended to cover the invention as broadly as legally possible in whatever form it may be utilized. The invention claimed herein is new and novel in accordance with 35 U.S.C. Section 102 and satisfies the conditions for patentability in Section 102. The invention claimed herein is not obvious in accordance with 35 U.S.C. Section 103 and satisfies the conditions for patentability in Section 103.

1.-70. (canceled)

71. A method for indicating a change in a coating, the coating containing nanomaterial, the method comprising
monitoring the coating with monitor apparatus, the monitor apparatus suitable for monitoring the nanomaterial to detect a change in the coating, the monitor apparatus including transmission apparatus for transmitting information about the change,
transmitting the information to analysis apparatus, and
with the analysis apparatus analyzing the information.

72. The method of claim 71 wherein the change in the coating is a result of one of a change in a parameter of the coating and a change in a condition of the coating.

73. The method of claim 71 wherein the change is a change in one of: electrical resistance of the coating, electrical conductivity of the coating, strain on the coating, configuration of the coating, cracking of the coating, dimensions of the coating, force on the coating, and temperature of the coating.

74. The method of claim 71 wherein the change is indicative of a change in pressure, gas presence, gas level, cracking, gas flow, pH, electromagnetic waves, force, liquid presence, fluid presence, liquid flow, fluid flow, presence of explosives, light, presence of light, intensity of light, crack growth, electrical conductivity, magnetic field strength, and electrochemical response.

75. The method of claim 71 wherein the monitor apparatus has a sensor element for sensing the change in the coating and the coating is on a thing, the method further comprising
sensing a change in the coating with the sensor element.

76. The method of claim 75 wherein the sensor element is on, within, outside of, or adjacent the thing with the coating.

77. The system of claim 75 wherein the thing is one of an airplane, wing, edge of a wing, propeller, turbine blade, turbine blade edge, tower, wind power generator, pipeline, pipeline interior, pipeline exterior, bridge, bridge deck, cable, support structure, ship, ship hull, boat, boat motor, vehicle, rail, automobile, truck, trailer, recreational vehicle, drilling system, pipe, tubular, casing, blowout preventer, seal, drilling equipment, drilling structure, drilling rig, conduit, offshore rig, centrifuge, shale shaker, screen, screen support, heat exchanger, finned tube, jet engine, tank, fabric, and cathodic protection structure.

78. The method of claim 75 wherein the thing is a tubular, the tubular having a threaded part, and the coating is on the threaded part.

79. The system of claim 75 wherein the thing is a tubular, the tubular has a shoulder, and the coating is on the shoulder.

80. The method of claim 75 wherein the coating is insulated from the tubular.

81. The method of claim 75 wherein the thing is a tubular; the tubular is one of conduit, coiled tubing, pipe, pipeline, drill pipe, casing, riser, and tubing; and, the method further comprising
sensing the change after use of the tubular.

82. The method of claim 75 wherein the thing comprises a first tubular threadedly connected to a second tubular, the method further comprising
sensing the change after the first tubular is threadedly connected to the second tubular.

83. The method of claim 71 wherein the nanomaterial is carbon nanotubes.

84. A tubular, the tubular comprising
a tubular body,
a coating on a part of the tubular body, the coating having nanomaterial therein,
the nanomaterial present in an effective amount for being monitored by a monitor apparatus for detecting a change in the coating.

85. The tubular of claim 84 wherein the tubular has a threaded part and the coating is on the threaded part.

86. The tubular of claim 84 wherein the tubular has a shoulder and the coating is on the shoulder.

87. The tubular of claim 84 wherein the tubular comprises a first tubular threadedly connected to a second.

88. The tubular of claim 84 wherein the coating is insulated from the tubular.

88. The tubular of claim 84 wherein the nanomaterial is carbon nanotubes.

90. A system for monitoring a thing, the thing having a coating, the coating having nanomaterial therein, the system comprising
monitoring apparatus for monitoring the coating with monitor apparatus, the monitor apparatus suitable for monitoring the nanomaterial to detect a change in the coating, the monitor apparatus including transmission apparatus for transmitting information about the change, and
analysis apparatus for analyzing the information.
